SEQUENCE LISTING (87 sequences)

SEQ ID NO: 1 - protein, 10 aa, Artificial Sequence
  VHCDR1
GNTFTSYVMH

SEQ ID NO: 2 - protein, 17 aa, Artificial Sequence
  VHCDR2
YINPYNDGTKYNEKFKG

SEQ ID NO: 3 - protein, 14 aa, Artificial Sequence
  VHCDR3
STARATPYFYAMDY

SEQ ID NO: 4 - protein, 17 aa, Artificial Sequence
  VLCDR1
KSSQSLLWSVNQNSYLS

SEQ ID NO: 5 - protein, 7 aa, Artificial Sequence
  VLCDR2
GASIRES

SEQ ID NO: 6 - protein, 11 aa, Artificial Sequence
  VLCDR3
QHNHGSFLPYT

SEQ ID NO: 7 - protein, 123 aa, Artificial Sequence
  AH1
QVQLVQSGVEVKKPGASVKVSCKASGNTFTSYVMHWVRQAPGQGLEWMAYINPYNDGTKYNEKFKGRVTLTTDSSTTTAYMELKSLQFDDTAVYYCARSTARATPYFYAMDYWGQGTLVTVSS

SEQ ID NO: 8 - protein, 123 aa, Artificial Sequence
  AH2
EVQLVESGGGLVQPGGSLRLSCAASGNTFTSYVMHWVRQAPGKGLEWVAYINPYNDGTKYNEKFKGRFTLSVDRSKNTLYLQMNSLRAEDTAVYYCARSTARATPYFYAMDYWGQGTLVTVSS

SEQ ID NO: 9 - protein, 123 aa, Artificial Sequence
  AH3
QVQLKQSGPGLVQPSQSLSITCTVSGNTFTSYVMHWVRQPPGRGLEWIAYINPYNDGTKYNEKFKGRVTMLVDTSKNQFSLRLSSVTAADTAVYYCARSTARATPYFYAMDYWGQGTLVTVSS

SEQ ID NO: 10 - protein, 123 aa, Artificial Sequence
  LPH1
QVQLVQSGAEVKKPGASVKVSCKASGNTFTSYVMHWVRQAPGQGLEWMGYINPYNDGTKYNEKFKGRVTMTRDTSTSTVYMELSSLRSEDTAVYYCARSTARATPYFYAMDYWGQGTLVTVSS

SEQ ID NO: 11 - protein, 123 aa, Artificial Sequence
  LPH2
EVQLVQSGAEVKKPGASVKVSCKASGNTFTSYVMHWVRQAPGQGLEWMGYINPYNDGTKYNEKFKGRVTMTSDKSTSTAYMELSSLRSEDTAVYYCARSTARATPYFYAMDYWGQGTLVTVSS

SEQ ID NO: 12 - protein, 123 aa, Artificial Sequence
  LPH4
EVQLVQSGAEVKKPGASVKVSCKASGNTFTSYVMHWVRQAPGQGLEWMGYINPYNDGTKYNEKFKGRVTITSDKSASTAYMELSSLRSEDTAVYYCARSTARATPYFYAMDYWGQGTLVTVSS

SEQ ID NO: 13 - protein, 115 aa, Artificial Sequence
  AL1
DIQMTQSPSSLSASVGDRVTITCKSSQSLLWSVNQNSYLSWYQQKPGKAPKLLIYGASIRESGVPSRFSGSGSGTDFTLTISSLQPEDFATYYCQHNHGSFLPYTFGQGTKVEIK

SEQ ID NO: 14 - protein, 115 aa, Artificial Sequence
  AL2
EIVLTQSPATLSLSPGERATLSCKSSQSLLWSVNQNSYLSWYQQKPGQAPRLLIYGASIRESGVPARFSGSGSGTDFTLTISSLEPEDFAVYYCQHNHGSFLPYTFGQGTKVEIK

SEQ ID NO: 15 - protein, 115 aa, Artificial Sequence
  AL3
DIVMTQSPDSLAVSLGERATINCKSSQSLLWSVNQNSYLSWYQQKPGQPPKLLIYGASIRESGVPDRFSGSGSGTDFTLTISSLQAEDVAVYYCQHNHGSFLPYTFGQGTKVEIK

SEQ ID NO: 16 - protein, 115 aa, Artificial Sequence
  LPL1
DIVMTQSPDSLAVSLGERATINCKSSQSLLWSVNQNSYLSWYQQKPGQPPKLLIYGASIRESGVPDRFSGSGSGTDFTLTISSLQAEDVAVYYCQHNHGSFLPYTFGQGTKLEIK

SEQ ID NO: 17 - protein, 115 aa, Artificial Sequence
  LPL2
DIVMTQSPDSLAVSLGERATINCKSSQSLLWSVNQNSYLSWYQLKPGQPPKLLLYGASIRESGVPDRFSGSGSGTDFTLTISSLQAEDVAVYYCQHNHGSFLPYTFGGGTKLEIK

SEQ ID NO: 18 - protein, 115 aa, Artificial Sequence
  LPL4
DILMTQSPSSLSASVGDRVTITCKSSQSLLWSVNQNSYLSWYQLKPGQPPKLLLYGASIRESGVPSRFSGSGSGTDFTLTISSLQPEDFATYYCQHNHGSFLPYTFGGGTKLEIK

SEQ ID NO: 19 - protein, 25 aa, Artificial Sequence
  VHFR1 sequence
QVQLVQSGVEVKKPGASVKVSCKAS

SEQ ID NO: 20 - protein, 25 aa, Artificial Sequence [sequence omitted]

SEQ ID NO: 21 - protein, 14 aa, Artificial Sequence
  VHFR2 sequence
WVRQAPGQGLEWMA

SEQ ID NO: 22 - protein, 14 aa, Artificial Sequence
  VHFR2 sequence
WVRQAPGQGLEWMG

SEQ ID NO: 23 - protein, 32 aa, Artificial Sequence
  VHFR3 sequence
RVTLTTDSSTTTAYMELKSLQFDDTAVYYCAR

SEQ ID NO: 24 - protein, 32 aa, Artificial Sequence
  VHFR3 sequence
RVTMTRDTSTSTVYMELSSLRSEDTAVYYCAR

SEQ ID NO: 25 - protein, 12 aa, Artificial Sequence
  VHFR4 sequence
YWGQGTLVTVSS

SEQ ID NO: 26 - protein, 23 aa, Artificial Sequence
  VLFR1 sequence
DIQMTQSPSSLSASVGDRVTITC

SEQ ID NO: 27 - protein, 23 aa, Artificial Sequence
  VLFR1 sequence
DIVMTQSPDSLAVSLGERATINC

SEQ ID NO: 28 - protein, 15 aa, Artificial Sequence
  VLFR2 sequence
WYQQKPGKAPKLLIY

SEQ ID NO: 29 - protein, 15 aa, Artificial Sequence
  VLFR2 sequence
WYQLKPGQPPKLLLY

SEQ ID NO: 30 - protein, 32 aa, Artificial Sequence
  VLFR3 sequence
GVPSRFSGSGSGTDFTLTISSLQPEDFATYYC

SEQ ID NO: 31 - protein, 32 aa, Artificial Sequence
  VLFR3 sequence
GVPDRFSGSGSGTDFTLTISSLQAEDVAVYYC

SEQ ID NO: 32 - protein, 10 aa, Artificial Sequence
  VLFR4 sequence
FGQGTKVEIK

SEQ ID NO: 33 - protein, 10 aa, Artificial Sequence
  VLFR4 sequence
FGGGTKLEIK

SEQ ID NO: 34 - protein, 25 aa, Artificial Sequence
  VHFR1 sequence
  MISC_FEATURE (9)..(9): wherein Xaa is V or A
QVQLVQSGXEVKKPGASVKVSCKAS

SEQ ID NO: 35 - protein, 14 aa, Artificial Sequence
  VHFR2 sequence
  MISC_FEATURE (14)..(14): wherein Xaa is A or G
WVRQAPGQGLEWMX

SEQ ID NO: 36 - protein, 25 aa, Artificial Sequence
  AH1 FR1
QVQLVQSGVEVKKPGASVKVSCKAS

SEQ ID NO: 37 - protein, 25 aa, Artificial Sequence [sequence omitted]

SEQ ID NO: 38 - protein, 25 aa, Artificial Sequence
  AH3 FR1
QVQLKQSGPGLVQPSQSLSITCTVS

SEQ ID NO: 39 - protein, 25 aa, Artificial Sequence [sequence omitted]

SEQ ID NO: 40 - protein, 25 aa, Artificial Sequence
  LPH2 FR1
EVQLVQSGAEVKKPGASVKVSCKAS

SEQ ID NO: 41 - protein, 25 aa, Artificial Sequence
  LPH4 FR1
EVQLVQSGAEVKKPGASVKVSCKAS

SEQ ID NO: 42 - protein, 14 aa, Artificial Sequence
  AH1 FR2
WVRQAPGQGLEWMA

SEQ ID NO: 43 - protein, 14 aa, Artificial Sequence
  AH2 FR2
WVRQAPGKGLEWVA

SEQ ID NO: 44 - protein, 14 aa, Artificial Sequence
  AH3 FR2
WVRQPPGRGLEWIA

SEQ ID NO: 45 - protein, 14 aa, Artificial Sequence
  LPH1 FR2
WVRQAPGQGLEWMG

SEQ ID NO: 46 - protein, 14 aa, Artificial Sequence
  LPH2 FR2
WVRQAPGQGLEWMG

SEQ ID NO: 47 - protein, 14 aa, Artificial Sequence
  LPH4 FR2
WVRQAPGQGLEWMG

SEQ ID NO: 48 - protein, 32 aa, Artificial Sequence; identical to SEQ ID NO: 23
  AH1 FR3

SEQ ID NO: 49 - protein, 32 aa, Artificial Sequence
  AH2 FR3
RFTLSVDRSKNTLYLQMNSLRAEDTAVYYCAR

SEQ ID NO: 50 - protein, 32 aa, Artificial Sequence
  AH3 FR3
RVTMLVDTSKNQFSLRLSSVTAADTAVYYCAR

SEQ ID NO: 51 - protein, 32 aa, Artificial Sequence; identical to SEQ ID NO: 24
  LPH1 FR3

SEQ ID NO: 52 - protein, 32 aa, Artificial Sequence
  LPH2 FR3
RVTMTSDKSTSTAYMELSSLRSEDTAVYYCAR

SEQ ID NO: 53 - protein, 32 aa, Artificial Sequence
  LPH4 FR3
RVTITSDKSASTAYMELSSLRSEDTAVYYCAR

SEQ ID NO: 54 - protein, 12 aa, Artificial Sequence
  AH1 FR4
YWGQGTLVTVSS

SEQ ID NO: 55 - protein, 12 aa, Artificial Sequence
  AH2 FR4
YWGQGTLVTVSS

SEQ ID NO: 56 - protein, 12 aa, Artificial Sequence
  AH3 FR4
YWGQGTLVTVSS

SEQ ID NO: 57 - protein, 12 aa, Artificial Sequence
  LPH1 FR4
YWGQGTLVTVSS

SEQ ID NO: 58 - protein, 12 aa, Artificial Sequence
  LPH2 FR4
YWGQGTLVTVSS

SEQ ID NO: 59 - protein, 12 aa, Artificial Sequence
  LPH4 FR4
YWGQGTLVTVSS

SEQ ID NO: 60 - protein, 23 aa, Artificial Sequence
  AL1 FR1
DIQMTQSPSSLSASVGDRVTITC

SEQ ID NO: 61 - protein, 23 aa, Artificial Sequence
  AL2 FR1
EIVLTQSPATLSLSPGERATLSC

SEQ ID NO: 62 - protein, 23 aa, Artificial Sequence
  AL3 FR1
DIVMTQSPDSLAVSLGERATINC

SEQ ID NO: 63 - protein, 23 aa, Artificial Sequence
  LPL1 FR1
DIVMTQSPDSLAVSLGERATINC

SEQ ID NO: 64 - protein, 23 aa, Artificial Sequence
  LPL2 FR1
DIVMTQSPDSLAVSLGERATINC

SEQ ID NO: 65 - protein, 23 aa, Artificial Sequence
  LPL4 FR1
DILMTQSPSSLSASVGDRVTITC

SEQ ID NO: 66 - protein, 15 aa, Artificial Sequence
  AL1 FR2
WYQQKPGKAPKLLIY

SEQ ID NO: 67 - protein, 15 aa, Artificial Sequence
  AL2 FR2
WYQQKPGQAPRLLIY

SEQ ID NO: 68 - protein, 15 aa, Artificial Sequence
  AL3 FR2
WYQQKPGQPPKLLIY

SEQ ID NO: 69 - protein, 15 aa, Artificial Sequence
  LPL1 FR2
WYQQKPGQPPKLLIY

SEQ ID NO: 70 - protein, 15 aa, Artificial Sequence
  LPL2 FR2
WYQLKPGQPPKLLLY

SEQ ID NO: 71 - protein, 15 aa, Artificial Sequence
  LPL4 FR2
WYQLKPGQPPKLLLY

SEQ ID NO: 72 - protein, 31 aa, Artificial Sequence
  AL1 FR3
VPSRFSGSGSGTDFTLTISSLQPEDFATYYC

SEQ ID NO: 73 - protein, 32 aa, Artificial Sequence
  AL2 FR3
GVPARFSGSGSGTDFTLTISSLEPEDFAVYYC

SEQ ID NO: 74 - protein, 32 aa, Artificial Sequence; identical to SEQ ID NO: 31
  AL3 FR3

SEQ ID NO: 75 - protein, 32 aa, Artificial Sequence; identical to SEQ ID NO: 31
  LPL1 FR3

SEQ ID NO: 76 - protein, 32 aa, Artificial Sequence; identical to SEQ ID NO: 31
  LPL2 FR3

SEQ ID NO: 77 - protein, 32 aa, Artificial Sequence; identical to SEQ ID NO: 30
  LPL4 FR3

SEQ ID NO: 78 - protein, 10 aa, Artificial Sequence
  AL1 FR4
FGQGTKVEIK

SEQ ID NO: 79 - protein, 10 aa, Artificial Sequence
  AL2 FR4
FGQGTKVEIK

SEQ ID NO: 80 - protein, 10 aa, Artificial Sequence
  AL3 FR4
FGQGTKVEIK

SEQ ID NO: 81 - protein, 10 aa, Artificial Sequence
  LPL1 FR4
FGQGTKLEIK

SEQ ID NO: 82 - protein, 10 aa, Artificial Sequence
  LPL2 FR4
FGGGTKLEIK

SEQ ID NO: 83 - protein, 10 aa, Artificial Sequence
  LPL4 FR4
FGGGTKLEIK

SEQ ID NO: 84 - protein, 222 aa, Artificial Sequence
  Light chain sequence
DIVMTQSPDSLAVSLGERATINCKSSQSLLWSVNQNSYLSWYQLKPGQPPKLLLYGASIRESGVPDRFSGSGSGTDFTLTISSLQAEDVAVYYCQHNHGSFLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 452 aa, Artificial Sequence
  Heavy chain sequence
QVQLVQSGAEVKKPGASVKVSCKASGNTFTSYVMHWVRQAPGQGLEWMGYINPYNDGTKYNEKFKGRVTMTRDTSTSTVYMELSSLRSEDTAVYYCARSTARATPYFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 702 aa, Artificial Sequence
  CEACAM5
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 87 - protein, 344 aa, Artificial Sequence
  CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

(19) United States
(12) Patent Application Publication
(10) Pub. No.: US 2024/0238433 A1
(43) Pub. Date: Jul. 18, 2024

(54) ANTI-CEACAM5/6 ANTIGEN-BINDING MOLECULES AND METHODS OF TREATMENT THEREOF

(71) Applicant: Agency for Science, Technology and Research, Singapore (SG)

(72) Inventors: Boon Hwa Andre CHOO, Singapore (SG); Ting Hwee GOH, Singapore (SG); Mei Yee DING, Singaore (SG); Hau Wan LEUNG, Singapore (SG); Simone DORFMUELLER, Singapore (SG); Ban Xiong TAN, Singapore (SG); Chek Shik LIM, Singapore (SG); Walter STUNKEL, Singapore (SG)

(21) Appl. No.: 18/562,788

(22) PCT Filed: May 20, 2022
(86) PCT No.: PCT/SG2022/050336

§ 371 Date: Nov. 20, 2023

(30) Foreign Application Priority Data
May 21, 2021 (SG) 10202105379W

(51) Int. Cl. A61K 47/68; A61P 35/00; C07K 16/28; G01N 33/574; G01N 33/58

(52) U.S. Cl. CPC A61K 47/68031; A61K 47/6849; A61P 35/00; C07K 16/2803; G01N 33/57492; G01N 33/58; C07K 2317/565; C07K 2317/567

(57) ABSTRACT

The invention relates to anti-CEACAM5/6 antigen-binding molecules and humanized variants thereof that bind to CEACAM5/6 that is glycosylated at N256. It also relates to the use of said antigen-binding molecules in methods of detection and medical treatment thereof.

### FIELD OF INVENTION

The invention relates generally to the field of antibody technology. In particular, the invention is directed to anti-CEACAM5/6 antigen-binding molecules and methods of treatment thereof.

### BACKGROUND

Carcinoembryonic antigen-related cell adhesion molecule 5 (CEACAM5) and carcinoembryonic antigen-related cell adhesion molecule 6 (CEACAM6) belong to the carcinoembryonic antigen (CEA) family. CEACAM5 and CEACAM6 are glycosyl phosphatidyl inositol (GPI) anchored cell surface glycoproteins which are known to be expressed highly in a wide variety of cancers that include gastric, breast, pancreatic, colon and non-small cell lung carcinoma (NSCL).

Post-translational glycosylation of CEACAMs is a cell-type and species dependent process that can alter the dimerization properties of CEACAMs. In oral squamous cell carcinoma, N-glycosylated CEACAM6 is a tumour marker associated with recurrence and is required for enhanced cell migration and invasion. N-terminally glycosylated CEACAM5 is upregulated in colorectal cancer (CRC). Furthermore, the protein expression of N-glycosylated CEACAM species is mostly found on the apical membranes of CRC cells, while being predominantly located inside normal colon cells.

While CEACAM5 and CEACAM6 are promising cancer targets, there are only a limited number of anti-CEACAM5 and anti-CEACAM6 monoclonal antibodies that are currently in development for cancer treatment in humans. There is therefore a need to develop new antibodies against these targets that are effective as cancer therapeutics.

Antibody humanization is a critical step in bringing an antibody to clinical therapeutic applications in humans. Antibody humanization of a mouse antibody involves replacing the mouse IgG backbone in the constant region and the framework regions in the variable region to the human versions, while retaining the original complementarity-determining regions (CDR) responsible for antigen binding. Antibody humanization reduces the risks of rejection and human anti-mouse antibody (HAMA) response that is known to occur with mouse-derived therapeutic antibodies. However, this process often impairs the original function and/or specificity of the antibody.

Accordingly, it is generally desirable to overcome or ameliorate one or more of the above mentioned difficulties.

### SUMMARY

Disclosed herein is an antigen-binding molecule comprising: (i) a heavy chain variable region (VH) comprising the VHCDR1 amino acid sequence of SEQ ID NO: 1, the VHCDR2 amino acid sequence of SEQ ID NO: 2, and the VHCDR3 amino acid sequence of SEQ ID NO: 3, and (ii) a light chain variable region (VL) comprising the VLCDR1 amino acid sequence of SEQ ID NO: 4, the VLCDR2 amino acid sequence of SEQ ID NO: 5, and the VLCDR3 amino acid sequence of SEQ ID NO: 6; wherein the VH as defined in (1) comprises at least 90% sequence identity to at least one region other than a CDR of the VH amino acid sequence set forth in SEQ ID NO: 10 or 7, and the VL as defined in (1) comprises at least 90% sequence identity to at least one region other than a CDR of the VL amino acid sequence set forth in SEQ ID NO: 17 or 13.

Disclosed herein is an isolated polynucleotide comprising a nucleic acid sequence encoding the antigen-binding molecule as defined herein.

Disclosed herein is a construct comprising a polynucleotide as defined herein in operable connection with one or more control sequence.

Disclosed herein is a host cell that contains the construct as defined herein.

Disclosed herein is a composition comprising an antigen-binding molecule as defined herein and a pharmaceutically acceptable carrier.

Disclosed herein is an antigen-binding molecule or a composition as defined herein for use as a medicament.

Disclosed herein is a method of treating or preventing a cancer or an inflammatory disease in a subject, the method comprising administering a therapeutically effective amount of an antigen-binding molecule or a composition as defined herein to the subject.

Disclosed herein is an antigen-binding molecule or a composition as defined herein for use in treating or preventing a cancer or an inflammatory disease in a subject.

Disclosed herein is the use of an antigen-binding molecule or a composition as defined herein in the manufacture of a medicament for treating or preventing a cancer or an inflammatory disease in a subject.

Disclosed herein is a method for detecting a cancer or an inflammatory disease in a subject, the method comprising: contacting a sample obtained from the subject with an antigen-binding molecule as defined herein, wherein an increase in the level of binding of the antigen-binding molecule in the sample as compared to a reference is indicative of a cancer or an inflammatory disease.

Disclosed herein is a method for identifying a subject susceptible to a cancer or an inflammatory disease, the method comprising: contacting a sample obtained from the subject with an antigen-binding molecule as defined herein, wherein an increase in the level of binding in the sample as compared to a reference indicates that the subject is susceptible to a cancer or an inflammatory disease.

Disclosed herein is a kit when used in the method as defined herein, comprising an antigen-binding molecule as defined herein, together with instructions for use.

### BRIEF DESCRIPTION OF DRAWINGS

Embodiments of the present invention are hereafter described, by way of non-limiting example only, with reference to the accompanying drawings in which:

FIG. 1 shows the potential variable heavy chain (VH) framework translated sequences with the CDR underlined.

FIG. 2 shows the potential variable light chain (VL) framework translated sequences with the CDR underlined.

FIG. 3: shows A) the Screening workflow and B) results of the Screening workflow.

FIG. 4: shows A) a summary of the screening outcome and B)-C) key changes in the variable heavy chain (VH) framework sequences and variable light chain (VL) framework sequences that led to decreased specificity highlighted in yellow. Variable heavy chains (VH) and variable light chains (VL) that preserved specificity are in italic.

FIG. 5: shows the heavy chain sequence and light chain sequence of the AB1 antibody. The AB1 antibody comprises the LPH1 VH and LPL2 VL sequences as shown in FIGS. 1 and 2. The AB2 antibody (not shown) comprises the AH1 VH and the AL1 VL sequences. Variable heavy chain (VH) and variable light chain (VL) are in bold with the CDR underlined.

FIG. 6 shows the translated sequence alignment of human CEACAM5 (amino acids 1-360 of SEQ ID NO: 86) and human CEACAM6 (amino acids 1-344 of SEQ ID NO: 87) with the conserved N256 glycosylation site.

FIG. 7 shows the characterization of the AB1 antigen. AB1 detect 2 bands in A549 cell lysate, a 75 kDa band and a 180 kDa band. A549 cells were subjected to single and double siRNA knock down of CEACAM5 and CEACAM6. (A) Western Blot analysis of siRNA treated cell lysate show that AB1 does not bind to the 180 kDa band in CEACAM5 single or double knock down samples. Further it is shown that AB1 does not bind to the 75 kDa band in CEACAM6 single or double knock down samples. AB1 detected the 75 kDa and 180 kDa band in scrambled gene siRNA knock down cell lysate and untreated cells (mock samples). CEACAM5 was knocked-down less efficiently than CEACAM6 in single as well as double knock down samples. Detection of the house keeping gene GAPDH serves as loading control. (B). Efficiency of siRNA knock down was monitored by gene expression analysis. CEACAM5 was knocked-down less efficiently than CEACAM6 in single as well as double knock down samples.

FIG. 8 shows N-glycosylation dependency of AB1 antigen recognition A549 cell lysate was reduced, denatured and treated with PNGase F to remove N-linked glycosylation and probed with AB1 and commercial CEACAM5 and CEACAM6 antibodies. Western Blot analysis shows that AB1 binding to the 75 kDa band (CEACAM6) and the 180 kDa band (CEACAM5) is abolished after reducing and after PNGase F treatment. Commercial CEACAM6 antibody detects reduced protein at 75 kDa and de-glycosylated CEACAM6 at lower size (37 kDa) whereas the commercial CEACAM5 antibody seems to be also N-glycan dependent as no de-glycosylated CEACAM5 is detected. Detection of the house keeping gene GAPDH serves as loading control.

FIG. 9 shows N256-glycosylation dependency of AB1 recognition. The lung cancer cell line NCI-H1299, which is not expressing CEACAM5 and CEACAM6 was transfected with CEACAM5 or CEACAM6 sequence and a CEACAM5 or CEACAM6 mutant sequence in which the N-glycosylation position 256 was mutated (N256A). This mutation abolishes N-glycosylation at position N256. Mean fluorescent intensity (MFI) is normalized (nMFI) against a human IgG1 isotype control (see Tables in FIGS. 9A and B). Flow cytometry binding of AB1 is compared to commercial CEACAM5 and CEACAM6 antibodies as well as competitor antibodies. Competitor antibodies included biosimilars to CEACAM6 specific Tinurilimab (Bayer), CEACAM5 specific Tusamitamab (Sanofi), N-glycosylated CEACAM5 and N-glycosylated CEACAM6 specific NEO-201 (Precision Biologics) and CEACAM5 and CEACAM6 specific EBC-123. EBC-123 is a biosimilar of L-DOS47 (Helix Biopharma Corp) whereby the original monomeric camelid single domain VHH 2A3 is engrafted on a human Fc. It is shown that AB1 does bind to NCI-H1299 cells expressing CEACAM5 or CEACAM6 (A), but not to cells expressing N256A mutated CEACAM5 or N256A mutated CEACAM6 (B). This indicates that N256 glycosylation of CEACAM5 and CEACAM6 is important for the binding of AB1 to CEACAM5 or CEACAM6. None of the CEACAM6 specific competitor antibodies (Tinurilimab, EBC-123 and NEO-201) showed this dependency on N256 glycosylation of CEACAM6 (FIG. 9A).

FIG. 10 shows flow cytometry binding of AB1, AB2 and AB3 to different populations of human primary peripheral blood cells (top) or human primary bone marrow leukocytes (below) in comparison to commercial and competitor antibodies. Primary cells with red blood cells lysed are incubated with different primary antibodies targeting CEACAM5 and CEACAM6. Cells are labelled with fluorescent-labelled linage markers for granulocytes (CD15+), T-cells (CD3+) or B-cells (CD19+). Mean fluorescent intensity (MFI, top) or normalized MFI (below) (right) is shown for 3 donor/2 repeats for primary peripheral blood cells and 1 donor/3 repeats for primary human bone marrow leukocytes. No (i.e. negligible) binding is seen to CD3⁺ T-cells by AB1, AB2 or AB3. AB1, AB2 and AB3 are found to bind less to granulocytes (CD15+ cell population) or B-cells (CD 19+ population) in the peripheral blood compared to CEACAM6 specific antibodies like Tinurilimab (Bayer), NEO-201 (Precision Biologics) and EBC-123 (Helix Biopharma Corp). The CEACAM5 specific Tusamitamab (Sanofi) shows no binding to human leukocytes.

FIG. 11 shows the affinity measurement by Bio-layer Interferometry on (A) CEACAM5 and N256A mutated CEACAM5 (as well as (B) CEACAM6 and N256A mutated CEACAM6. In-house produced Avi-tagged CEACAM5, CEACAM6 and N256A mutated CEACAM5 and N256A mutated CEACAM6 are immobilized to a Dip and Read SA biosensor. The antibody AB1 and competitor antibodies EBC-123 (Helix Biopharma Corp), NEO-201 (Precision Biologics) and Tusamitamab (Sanofi) are used as analyte. Results show that the AB1 binds to both CEACAM5 and CECAM6 with comparable affinity constants (KDs) in the 2 digit nanomolar range. KDs for the AB1 are 10-100-fold smaller on N256A mutated CEACAM5 and N256A mutated CEACAM6 compared to wildtype CEACAM5 and CEACAM6 protein. The competitor antibodies bind to CEACAM5, CEACAM6, N256A mutated CEACAM5 and N256A mutated CEACAM6 with similar affinity.

FIG. 12 shows the internalization of the antibody AB1 and competitor antibodies into antigen positive and antigen negative cell lines. A) shows internalization of AB1 in NCI-H1299 and NCI-H1299 overexpressing CEACAM5 or CEACAM6 or N256A mutated CEACAM5 or N256A mutated CEACAM6. B) shows internalization of AB1 in comparison to the IgG1 isotype control and competitor antibodies specific to CEACAM5 and/or CEACAM6 into NCI-H1299 and NCI-H1299 overexpressing CEACAM5 or CEACAM6 cells. AB1 internalises in NCI-H1299 expressing CEACAM5 or CEACAM6, but not NCI-H1299 or NCI-H1299 expressing N256A mutated CEACAM5 or N256A mutated CEACAM6 (A). CEACAM6 specific Tinurilimab (Bayer) internalises non-specifically into NCI-H1299 cells. CEACAM5 specific Tusamitamab (Sanofi) internalizes only into NCI-H1299 overexpressing CEACAM5 while the CEACAM5 and CEACAM6 specific NEO-201 (Precision Biologics) and EBC-123 (Helix Biopharma Corp) internalise at comparable rates as AB1 into NCI-H1299 overexpressing CEACAM5 or CEACAM6 (B).

FIG. 13 shows the conjugated antibody AB3. AB3 consists of AB1 that is conjugated to monomethyl auristatin E (MMAE) via a protease-cleavable maleimidocaproyl valine citrulline (vc-PAB) linker

FIG. 14 shows in vitro functionality by CellTiterGlo™ viability assay and dose-response curves estimating IC₅₀ after incubation of cells for 72h. A) left panel shows dose-response curved of AB3 with NCI-H1299 in comparison with NCI-H1299 overexpressing CEACAM6 (clonal cell line 12) or the mutated N256A CEACAM6 (clonal cell line 19). A) right panel shows dose-response curved of AB3 with NCI-H1299 in comparison with NCI-H1299 overexpressing CEACAM5 (clonal cell line 2F3) or the mutated N256A CEACAM5 (clonal cell line 6). B) shows IC₅₀ obtained with AB3, IgG1 isotype control-MMAE and free MMAE with NCI-H1299 cells and NCI-H1299 cells overexpressing CEACAM5, CEACAM6, N256A mutated CEACAM5 and N256A mutated CEACAM6. 2-digit nanomolar IC₅₀ are achieve for NCI-H1299 cells that express membrane bound CEACAM5 or CEACAM6 whereas higher IC₅₀ are obtained for NCI-H1299 cells not expressing CEACAM5 nor CEACAM6 and NCI-H1299 cells expressing the N256A mutated CEACAM5 or N256A mutated CEACAM6. Comparable high IC₅₀ are achieved with the IgG1-MMAE isotype control for all cell lines, demonstrating the specificity of AB3. Free MMAE has an IC₅₀ in the picomolar range. C) show IC₅₀ with AB3 in comparison to the CEACAM5 specific competitor antibody Tusamitamab (Sanofi) conjugated to MMAE. IC₅₀ of both AB3 and Tusamitamab-MMAE are compared for the cancer cell lines Capan-1, CFPAC-1, HCC4006, SNU-16 and HT-29. AB3 target a wider range of cancer indications due to its dual specificity for N256 glycosylated CEACAM5 and N256 glycosylated CEACAM6.

FIG. 15 shows AB3 in a gastric xenograft model. SNU-16 cells expresses both AB1 antigens CEACAM5 and CEACAM6. Female Balb/c nude mice (n=10/group) are implanted with SNU-16 cells and treated once IV (or once weekly x3 for docetaxel). AB3 is given at 1, 3 or 5 mg/kg with only a single administration on Day 0. AB3 achieves TGIs of 154%, 147% and 114% respectively on Day 35. Treatment is very well tolerated with no weight loss observed. Treatment with IgG1-MMAE at 5 or 1 mg/kg has statistical significantly (p=0.05) lower TGI (123% and 95% respectively). Furthermore, only AB3 treated mice show complete responses in a dose dependent manner, with 6/10, 3/10 and 1/10 mice being tumour free after a single dose of 5, 3 and 1 mg/kg AB3 respectively, but no tumour free animals are observed after treatment with IgG1-MMAE only. Anova: One-way ANOVA test followed by Bonferroni Multiple Comparison Test, significance levels shown compared to G1: p<0.0001=****, p<0.001=**, p<0.05=*, ns=Not Significant; NA=Not applicable; BW, Body-weight (100% set per animal at day 0/inoculation, group means in % are shown); MTV, Mean Tumor Volume of all/remaining animals; n=number; PR, Partial Regression; CR, Complete Regression; TGI, Tumour Growth Inhibition=Mean Tumour Volume (Control Day 35−Treated Day 35)/(Control Day 21−Control Day 0); TRD, Treatment-Related Death; NTRD, Non-Treatment-Related Death (e.g. ulcerated tumour, dosing error).

FIG. 16 shows AB3 in a pancreatic xenograft model Capan-1. Capan-1 cells expresses both AB1 antigens CEACAM5 and CEACAM6. Female NSG mice (n=10/group) are implanted with Capan-1 cells and treated once IV. AB3 is given at 5 mg/kg with only a single administration on Day 0. AB3 achieves TGIs of 110% on Day 21. Treatment is very well tolerated with no weight loss observed. Treatment with IgG1-MMAE at 5, 3 or 1 mg/kg has statistical significantly (p=0.05) lower TGI (28%, 185 and −8% respectively). Anova: One-way ANOVA test followed by Bonferroni Multiple Comparison Test, significance levels shown compared to G1: p<0.0001=****, p<0.001=**, p<0.05=*, ns=Not Significant; BW, Body-weight (100% set per animal at day 0/inoculation, group means in % are shown); MTV, Mean Tumor Volume of all/remaining animals; n=number; PR, Partial Regression; CR, Complete Regression; TGI, Tumour Growth Inhibition=Mean Tumour Volume (Control Day 21−Treated Day 21)/(Control Day 21−Control Day 0); TRD, Treatment-Related Death; NTRD, Non-Treatment-Related Death (e.g. ulcerated tumour, dosing error).

FIG. 17 shows AB3 in a pancreatic xenograft model BxPC-3. BxPC-3 cells expresses both AB1 antigens CEACAM5 and CEACAM6. Female NOD-SCID mice (n=8/group) are implanted with BxPC-3 cells and treated once IV. AB3 is given at 1 or 5 mg/kg with only a single administration on Day 0. AB3 achieves TGIs of 107% and 21% respectively on Day 21. Treatment is very well tolerated with no weight loss observed. Treatment with IgG1-MMAE at 5 mg/kg has statistical significantly (p=0.05) lower TGI of 6%. Anova: One-way ANOVA test followed by Bonferroni Multiple Comparison Test, significance levels shown compared to G1: p<0.0001=****, p<0.001=**, p<0.05=*, ns=Not Significant; BW, Body-weight (100% set per animal at day 0/inoculation, group means in % are shown); MTV, Mean Tumor Volume of all/remaining animals; n=number; PR, Partial Regression; CR, Complete Regression; TGI, Tumour Growth Inhibition=Mean Tumour Volume (Control Day 21−Treated Day 21)/(Control Day 21−Control Day 0); TRD, Treatment-Related Death; NTRD, Non-Treatment-Related Death (e.g. ulcerated tumour, dosing error).

FIG. 18 shows tumour growth profiles of antigen negative NCI-H1299 and NCI-H1299 overexpressing N256A mutated CEACAM5 or N256A mutated CEACAM6 versus antigen positive NCI-H1299 overexpressing CEACAM5 or CEACAM6. Female NOD-SCID mice (n=6/group) are implanted with NCI-H1299 cells and tumour volumes are measured 3 times per week up to 75 days. No difference in tumour onset or doubling time is observed for NCI-H299, NCI-H1299 overexpressing CEACAM6 or NCI-H1299 overexpressing N256A mutated CEACAM6. Early tumour onset is observed for NCI-H1299 overexpressing CEACAM5, while tumour onset for NCI-H1299 overexpressing N256A mutated CEACAM5 is delayed by 35 days. After tumour onset the tumour doubling time is comparable for all cell lines.

FIG. 19 shows AB3 in a xenograft model using antigen negative NCI-H1299 and NCI-H1299 overexpressing N256A mutated CEACAM5 versus antigen positive NCI-H1299 overexpressing CEACAM5 or CEACAM6 cells. Female NOD-SCID mice (n=6/group) are implanted with NCI-H1299 cells and treated once IV. AB3 is given at 5 mg/kg with only a single administration on Day 0. AB3 achieves TGIs of 32% and 28% respectively for antigen negative cells NCI-H1299 and NCI-H1299 overexpressing N256A mutated CEACAM5 on Day 21. AB3 achieves TGIs of 109% and 110% respectively for antigen positive cells NCI-H1299 overexpressing CEACAM5 or CEACAM6. Treatment is very well tolerated with no weight loss observed. Anova: One-way ANOVA test followed by Bonferroni Multiple Comparison Test, significance levels shown compared to G1: p<0.0001=****, p<0.001=**, p<0.05=*, ns=Not Significant; BW, Body-weight (100% set per animal at day 0/inoculation, group means in % are shown); MTV, Mean Tumor Volume of all/remaining animals; n=number; PR, Partial Regression; CR, Complete Regression; TGI, Tumour Growth Inhibition=Mean Tumour Volume (Control Day 21−Treated Day 21)/(Control Day 21−Control Day 0); TRD, Treatment-Related Death; NTRD, Non-Treatment-Related Death (e.g. ulcerated tumour, dosing error).

### DETAILED DESCRIPTION

The present specification teaches a humanized antigen-binding molecule that binds to CEACAM 5 and/or CEACAM6. The specification teaches a humanized antigen-binding molecule comprising a (i) heavy chain variable region (VH) comprising the VHCDR1 amino acid sequence of GNTFTSYVMH (SEQ ID NO: 1), the VHCDR2 amino acid sequence of YINPYNDGTKYNEKFKG (SEQ ID NO: 2) and the VHCDR3 amino acid sequence of STARATPYFYAMDY (SEQ ID NO: 3); and (ii) a light chain variable region (VL) comprising the VLCDR1 amino acid sequence of KSSQSLLWSVNQNSYLS (SEQ ID NO: 4), the VLCDR2 amino acid sequence of GASIRES (SEQ ID NO: 5), and the VLCDR3 amino acid sequence of QHNHGSFLPYT (SEQ ID NO: 6).

Disclosed herein is an antigen-binding molecule, comprising: (1) a heavy chain variable region (VH) comprising the VHCDR1 amino acid sequence of SEQ ID NO: 1, the VHCDR2 amino acid sequence of SEQ ID NO: 2, and the VHCDR3 amino acid sequence of SEQ ID NO: 3, and a light chain variable region (VL) comprising the VLCDR1 amino acid sequence of SEQ ID NO: 4, the VLCDR2 amino acid sequence of SEQ ID NO: 5, and the VLCDR3 amino acid sequence of SEQ ID NO: 6; wherein the VH as defined in (1) comprises at least 90% (including at least 91% to 99% and all integer percentages therebetween) sequence identity to at least one region other than a CDR of the VH amino acid sequence set forth in SEQ ID NO: 10 or 7, and the VL as defined in (1) comprises at least 90% (including at least 91% to 99% and all integer percentages therebetween) sequence identity to at least one region other than a CDR of the VL amino acid sequence set forth in SEQ ID NO: 17 or 13.

Without being bound by theory, the inventors have found that during the process of converting the murine antibody to a humanized antibody, many variants showed decreased specificity and/or function compared to the parental murine and chimeric forms. Several key criteria were identified to rank the humanized variants resultant from in silico humanization optimisation. Importantly, it was the aggregation and site specificity read-outs that enabled the elimination of problematic motifs, and enabled rapid short-listing of three final molecules for further pre-clinical evaluation.

In one embodiment, the antigen-binding molecule binds to CEACAM5 and/or CEACAM6. In one embodiment, the antigen-binding molecule binds to CEACAM5. In one embodiment, the antigen-binding molecule binds to CEACAM6. In one embodiment, the antigen-binding molecule binds to CEACAM5 and CEACAM6.

In one embodiment, the antigen-binding molecule binds to CEACAM5 and/or CEACAM6 when glycosylated on the N256 position. In one embodiment, the antigen-binding molecule binds to CEACAM5 when CEACAM5 is glycosylated on the N256 position. In one embodiment, the antigen-binding molecule binds to CEACAM6 when CEACAM6 is glycosylated on the N256 position. In one embodiment, the antigen-binding molecule binds to CEACAM5 and CEACAM6 when both CEACAM5 and CEACAM6 are glycosylated on the N256 position.

The antigen-binding molecules of the present invention may be in isolated, purified, synthetic or recombinant form. Suitable antigen-binding molecules may be selected from antibodies and their antigen-binding fragments, including monoclonal antibodies (MAbs), chimeric antibodies, humanized antibodies, human antibodies, and antigen-binding fragments of such antibodies. The antigen-binding molecules may be multivalent (e.g., bivalent) or monovalent. In some embodiments, the antigen-binding molecules comprise an Fc domain. In other embodiments, the antigen-binding molecules lack an Fc domain. In some embodiments, the antigen binding molecules are monovalent antigen-binding molecules (e.g., Fab, scFab, Fab′, scFv, one-armed antibodies, etc.).

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

The term “antibody”, as used herein, is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulphide bonds, as well as multimers thereof (e.g., IgM). Each heavy chain comprises a heavy chain variable region (which may be abbreviated as HCVR, VH) and a heavy chain constant region. The heavy chain constant region typically comprises three domains—CH1, CH2 and CH3. Each light chain comprises a light chain variable region (which may be abbreviated as LCVR or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR). Each VH and VL typically comprises three CDRs and four FRs, arranged from amino-terminus to carboxy-terminus in the following order: FR1, CDR1, FR2, CDR2, FR3, CDR3, FR4. In some embodiments, the FRs of the antigen-binding molecules described herein may be identical to the FR of germline sequences of the target species (i.e., the species to which the antigen-binding molecules or antigen-binding fragments thereof, as described herein, will be administered). In some embodiments, the FR may be naturally or artificially modified. Whilst it is generally desirable that each of the FR sequences are identical to FR sequences derived from immunoglobulin molecules of the target species, including to minimize an immune response being raised against the binding molecule upon administration to a subject of the target species, in some embodiments, the antigen-binding molecule, or antigen-binding fragment thereof, may comprise one or more amino acid residues across one or more of its FR sequences that would be foreign at a corresponding position in one or more FR from the target species

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known to a person skilled in the art. The light chains can be divided into kappa and lambda light chains.

In one embodiment, the antigen-binding molecule of the present invention has an isotype selected from the group consisting of IgG1, IgG2, IgG3, and IgG4. The heavy chain constant region can be a wild-type human Fc region, or a human Fc region that includes one or more amino acid substitutions. The antibodies can have mutations that stabilize the disulphide bond between the two heavy chains of an immunoglobulin, such as mutations in the hinge region of IgG4, as disclosed in the art (e.g., Angal et al., 1993. Mol. Immunol., 30:105-08). See also, e.g., U.S. 2005/0037000. The heavy chain constant region can also have substitutions that modify the properties of the antigen-binding molecule (e.g., decrease one or more of: Fc receptor binding, antigen-binding molecule glycosylation, deamidation, binding to complement, or methionine oxidation). In some instances, the antigen-binding molecules may have mutations such as those described in U.S. Pat. Nos. 5,624,821 and 5,648,260. In some embodiments, the antigen-binding molecule is modified to reduce or eliminate effector function. The heavy chain constant region can be chimeric, e.g., the Fc region can comprise the CH1 and CH2 domains of an IgG antibody of the IgG4 isotype, and the CH3 domain from an IgG antibody of the IgG1 isotype (see, e.g., U.S. Patent Appl. No. 2012/0100140A1).

As used herein, the term “complementarity determining regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined for example by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

An “antigen-binding site” (or paratope) refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site. An antigen-binding site of an antigen-binding molecule described herein typically binds specifically to an antigen and more particularly to an epitope of the antigen.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), camelid VHH and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3, (vi) VH-CH2-CH3; (vii) VH-CL; (viii) VL-CH1; (ix) VL-CH2, (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulphide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (or CDRs). See, e.g., Kindt et al., Kuby Immunology, 6th ed., W.H. Freeman and Co., page 91 (2007). A single VH or VL domain may be sufficient to confer antigen-binding specificity.

The term “constant domains” or “constant region” as used herein denotes the sum of the domains of an antibody other than the variable region. The constant region is not directly involved in binding of an antigen but exhibits various immune effector functions.

In an embodiment, the antigen-binding molecule or antigen-binding fragment thereof is humanized. By “humanized” is meant that the antigen-binding molecule comprises an amino acid sequence that is compatible with humans, such that the amino acid sequence is unlikely to be seen as foreign by the immune system of a human subject. In an embodiment, the humanized antigen-binding molecule comprises one or more immunoglobulin framework regions derived from one or more human immunoglobulin molecules. In some embodiments, all of the framework regions of the humanized antigen-binding molecule will be derived from one or more human immunoglobulin molecules. The humanized antibody may optionally comprise at least a portion of an antibody constant region derived from a human immunoglobulin molecule.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976)).

“Affinity” or “binding affinity” refers to the strength of the sum total of non-covalent interactions between a single binding site of a molecule (e.g., an antigen-binding molecule) and its binding partner (e.g., an antigen). Unless indicated otherwise, as used herein, “binding affinity” refers to intrinsic binding affinity which reflects a 1:1 interaction between members of a binding pair e.g., an antigen-binding molecule. The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR). The affinity can also be measured using Bio-layer Interferometry (BLI). In another embodiment, the method for measuring affinity is determined using a cell-based affinity measurement technique.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide

As used herein, the term “modified antibody” includes synthetic forms of antibodies which are altered such that they are not naturally occurring, e.g., antibodies that comprise at least two heavy chain portions but not two complete heavy chains (such as domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

In one embodiment, a) the VH as defined in (1) comprises at least 90% sequence identity to at least one region other than a CDR of the VH amino acid sequence set forth in SEQ ID NO: 10, and the VL as defined in (1) comprises at least 90% sequence identity to at least one region other than a CDR of the VL amino acid sequence set forth in SEQ ID NO: 17; b) the VH as defined in (1) comprises at least 90% sequence identity to at least one region other than a CDR of the VH amino acid sequence set forth in SEQ ID NO: 10, and the VL as defined in (1) comprises at least 90% sequence identity to at least one region other than a CDR of the VL amino acid sequence set forth in SEQ ID NO: 13; c) the VH as defined in (1) comprises at least 90% sequence identity to at least one region other than a CDR of the VH amino acid sequence set forth in SEQ ID NO: 7, and the VL as defined in (1) comprises at least 90% sequence identity to at least one region other than a CDR of the VL amino acid sequence set forth in SEQ ID NO: 17; or d) the VH as defined in (1) comprises at least 90% sequence identity to at least one region other than a CDR of the VH amino acid sequence set forth in SEQ ID NO: 7, and the VL as defined in (1) comprises at least 90% sequence identity to at least one region other than a CDR of the VL amino acid sequence set forth in SEQ ID NO: 13.

In one embodiment, a) the VH is distinguished from the VH amino acid sequence set forth in SEQ ID NO: 10 by a deletion, substitution or addition of one or more amino acids (e.g., 1, 2, 3, 4 or 5) in at least one region other than a CDR of the VH amino acid sequence set forth in SEQ ID NO: 10, and the VL is distinguished from the VL amino acid sequence set forth in SEQ ID NO: 17 by a deletion, substitution or addition of one or more amino acids (e.g., 1, 2, 3, 4 or 5) in at least one region other than a CDR of the VL amino acid sequence set forth in SEQ ID NO: 17; b) the VH is distinguished from the VH amino acid sequence set forth in SEQ ID NO: 10 by a deletion, substitution or addition of one or more amino acids (e.g., 1, 2, 3, 4 or 5) in at least one region other than a CDR of the VH amino acid sequence set forth in SEQ ID NO: 10, and the VL is distinguished from the VL amino acid sequence set forth in SEQ ID NO: 13 by a deletion, substitution or addition of one or more amino acids (e.g., 1, 2, 3, 4 or 5) in at least one region other than a CDR of the VL amino acid sequence set forth in SEQ ID NO: 13; c) the VH is distinguished from the VH amino acid sequence set forth in SEQ ID NO: 7 by a deletion, substitution or addition of one or more amino acids (e.g., 1, 2, 3, 4 or 5) in at least one region other than a CDR of the VH amino acid sequence set forth in SEQ ID NO: 7, and the VL is distinguished from the VL amino acid sequence set forth in SEQ ID NO: 17 by a deletion, substitution or addition of one or more amino acids (e.g., 1, 2, 3, 4 or 5) in at least one region other than a CDR of the VL amino acid sequence set forth in SEQ ID NO: 17; or d) the VH is distinguished from the VH amino acid sequence set forth in SEQ ID NO: 7 by a deletion, substitution or addition of one or more amino acids (e.g., 1, 2, 3, 4 or 5) in at least one region other than a CDR of the VH amino acid sequence set forth in SEQ ID NO: 7, and the VL is distinguished from the VL amino acid sequence set forth in SEQ ID NO: 13 by a deletion, substitution or addition of one or more amino acids (e.g., 1, 2, 3, 4 or 5) in at least one region other than a CDR of the VL amino acid sequence set forth in SEQ ID NO: 13.

In one embodiment, the antigen-binding molecule comprises:
a) a VHFR1 that is distinguished from a VHFR1 amino acid sequence set forth in QVQLVQSGVEVKKPGASVKVSCKAS (SEQ ID NO: 19) or QVQLVQSGAEVKKPGASVKVSCKAS (SEQ ID NO: 20) by a deletion, substitution or addition of one or more amino acids;
b) a VHFR2 that is distinguished from a VHFR2 amino acid sequence set forth in WVRQAPGQGLEWMA (SEQ ID NO: 21) or WVRQAPGQGLEWMG (SEQ ID NO: 22) by a deletion, substitution or addition of one or more amino acids;
c) a VHFR3 that is distinguished from a VHFR3 amino acid sequence set forth in RVTLTTDSSTTTAYMELKSLQFDDTAVYYCAR (SEQ ID NO: 23) or RVTMTRDTSTSTVYMELSSLRSEDTAVYYCAR (SEQ ID NO: 24) by a deletion, substitution or addition of one or more amino acids;
d) a VHFR4 that is distinguished from a VHFR4 amino acid sequence set forth in YWGQGTLVTVSS (SEQ ID NO: 25) by a deletion, substitution or addition of one or more amino acids;
e) a VLFR1 that is distinguished from a VLFR1 amino acid sequence set forth in DIQMTQSPSSLSASVGDRVTITC (SEQ ID NO: 26) or DIVMTQSPDSLAVSLGERATINC (SEQ ID NO: 27) by a deletion, substitution or addition of one or more amino acids;
f) a VLFR2 that is distinguished from a VLFR2 amino acid sequence set forth in WYQQKPGKAPKLLIY (SEQ ID NO: 28) or WYQLKPGQPPKLLLY (SEQ ID NO: 29) by a deletion, substitution or addition of one or more amino acids;
g) a VLFR3 that is distinguished from a VLFR3 amino acid sequence set forth in GVPSRFSGSGSGTDFTLTISSLQPEDFATYYC (SEQ ID NO: 30) or GVPDRFSGSGSGTDFTLTISSLQAEDVAVYYC (SEQ ID NO: 31) by a deletion, substitution or addition of one or more amino acids; and/or
h) a VLFR4 that is distinguished from a VLFR4 amino acid sequence set forth in FGQGTKVEIK (SEQ ID NO: 32) or FGGGTKLEIK (SEQ ID NO: 33) by a deletion, substitution or addition of one or more amino acids.

In one embodiment, the antigen-binding molecule comprises:
a) a VHFR1 amino acid sequence of QVQLVQSGX₁EVKKPGASVKVSCKAS, wherein X₁ is V or A (SEQ ID NO: 34);
b) a VHFR2 amino acid sequence of WVRQAPGQGLEWMX₂, wherein X₂ is A or G (SEQ ID NO: 35);
c) a VHFR3 amino acid sequence of RVTLTTDSSTTTAYMELKSLQFDDTAVYYCAR (SEQ ID NO: 23) or RVTMTRDTSTSTVYMELSSLRSEDTAVYYCAR (SEQ ID NO: 24);
d) a VHFR4 amino acid sequence of YWGQGTLVTVSS (SEQ ID NO: 25);
e) a VLFR1 amino acid sequence of DIQMTQSPSSLSASVGDRVTITC (SEQ ID NO: 26) or DIVMTQSPDSLAVSLGERATINC (SEQ ID NO: 27);
f) a VLFR2 amino acid sequence of WYQQKPGKAPKLLIY (SEQ ID NO: 28) or WYQLKPGQPPKLLLY (SEQ ID NO: 29);
g) a VLFR3 amino acid sequence of GVPSRFSGSGSGTDFTLTISSLQPEDFATYYC (SEQ ID NO: 30) or GVPDRFSGSGSGTDFTLTISSLQAEDVAVYYC (SEQ ID NO: 31); and/or
h) a VLFR4 amino acid sequence of FGQGTKVEIK (SEQ ID NO: 32) or FGGGTKLEIK (SEQ ID NO: 33).

In one embodiment, the antigen-binding molecule comprises a VH amino acid sequence of SEQ ID NO: 10 or 7 and a VL amino acid sequence of SEQ ID NO: 17 or 13.

In one embodiment, a) the antigen-binding molecule comprises a VH amino acid sequence of SEQ ID NO: 10 and a VL amino acid sequence of SEQ ID NO: 17; b) the antigen-binding molecule comprises a VH amino acid sequence of SEQ ID NO: 10 and a VL amino acid sequence of SEQ ID NO: 13; c) the antigen-binding molecule comprises a VH amino acid sequence of SEQ ID NO: 7 and a VL amino acid sequence of SEQ ID NO: 17; or d) the antigen-binding molecule comprises a VH amino acid sequence of SEQ ID NO: 7 and a VL amino acid sequence of SEQ ID NO: 13.

In one embodiment, the antigen-binding molecule is an antibody or antigen-binding fragment thereof. In one embodiment, the antibody or antigen binding fragment thereof is a full-length antibody, a substantially intact antibody, a Fab fragment, a scFab, a Fab′, a single chain variable fragment (scFv) or a one-armed antibody.

In one embodiment, the antigen-binding molecule is a full-length antibody. In one embodiment, the full-length antibody is an IgG (such as an IgG1) antibody.

In one embodiment, the antigen-binding molecule comprises a light chain sequence having at least 70% (including at least 71% to 99% and all integer percentages therebetween) sequence identity to SEQ ID NO: 84, and a heavy chain sequence having at least 70% (including at least 71% to 99% and all integer percentages therebetween) sequence identity to SEQ ID NO: 85.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G and I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The antigen-binding molecule as defined herein may comprise one or more conservative amino acid substitutions.

A “conservative amino acid substitution” is to be understood as meaning a substitution in which the amino acid residue is replaced with an amino acid residue having a similar side chain. Families of amino acid residues having similar side chains have been defined in the art, which can be generally sub-classified as shown in the table “Amino Acid Classification”, below:

AMINO ACID SUB-CLASSIFICATION

| Sub-classes | Amino acids |
|---|---|
| Acidic | Aspartic acid, Glutamic acid |
| Basic | Arginine, Lysine, Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Conservative substitutions are also shown in the table below (EXEMPLARY AND PREFERRED AMINO ACID SUBSTITUTIONS). Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants can be screened for their ability to bind specifically to the antigenusing methods known to persons skilled in the art, including those methods described elsewhere herein.

EXEMPLARY AND PREFERRED
AMINO ACID SUBSTITUTIONS

| Original Residue | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe | Leu |
| Leu | Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

In one embodiment, the antigen-binding molecule is conjugated to a radioisotope or cytotoxin.

In one embodiment, the antigen-binding molecule is a full-length IgG (such as IgG1) antibody conjugated to a cytotoxin. The antigen-binding molecule may be conjugated to the cytotoxin via a protease-cleavable maleimidocaproyl valine citrulline (vc-PAB) linker.

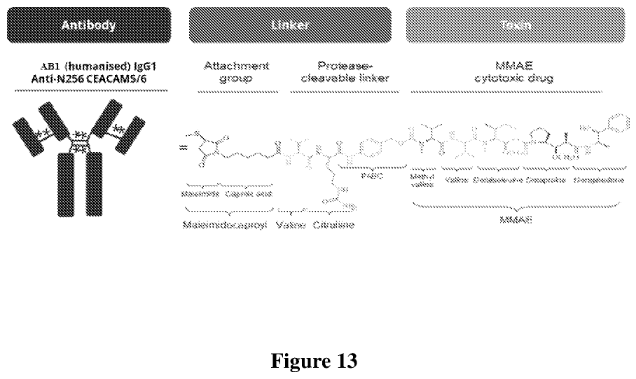

In one embodiment, the antigen-binding molecule consists of AB1 that is conjugated to monomethyl auristatin E (MMAE) via a maleimidocaproyl valine citrulline (vc-PAB) linker (i.e. AB3 as shown in FIG. 13). In one embodiment, the linker-toxin combination has the chemical formula of C₅₈H₉₄N₁₀O₁₂ with a chemical name of L-Valinamide, N-methyl-N-[[4-[[L-valyl-N5-(aminocarbonyl)-L-ornithyl]amino]phenyl] methoxy]carbonyl]-L-valyl-N-[(1S,2R)-4-[(2S)-2-[(1R,2R)-3-[[(1R,2S)-2-hydroxy-1-methyl-2-phenylethyl] amino]-1-methoxy-2-methyl-3-oxopropyl]-1-pyrrolidinyl]-2-methoxy-1-[(1S)-1-methylpropyl]-4-oxobutyl]-N-methyl. The conjugation may be performed by a maleimide-cysteine based method by first reducing the mAb inter-chain disulfide bonds with TCEP at 37° C. and then linking the maleimide moiety of the drug to the reduce cysteines. Drug Antibody Ratio (DAR) may be analysed on hydrophobic interaction chromatography (HIC). The DAR ratio may, for example, be between 3 and 4.

In another aspect, there is provided an antigen-binding molecule or antigen-drug conjugate (ADC) of the following formula (I):

Ab-(-D)n (I)
or a pharmaceutically acceptable salt thereof,
wherein
Ab is an antibody or antibody fragment thereof as defined herein:
L is a linker;
D is a cytotoxin.

In an embodiment of the invention relates to an ADC wherein L is a linker of the following formula (II):

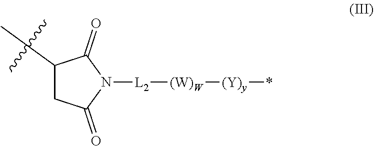

wherein
L2 is cycloalkylene-carbonyl, (C2-C6)alkyl or (C2-C6)alkyl-carbonyl;
W is an amino acid unit; w is an integer comprised of 0 to 5;
Y is PAB-carbonyl with PAB being

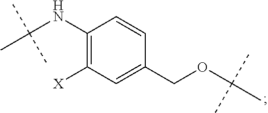

x can be H or

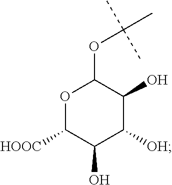

y is 0 or 1;
the asterisk indicates the point of attachment to D; and
the wavy line indicates the point of attachment to Ab.

An embodiment of the invention relates to an ADC wherein L2 is of the following formula:

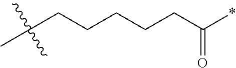 [SMILES: *CCCCCC(*)=O]

wherein
the asterisk indicates the point of attachment to (W)_{w}; and
the wavy line indicates the point of attachment to the nitrogen atom of the maleimide moiety of formula:

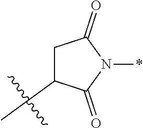 [SMILES: *C1CC(=O)N(*)C1=O]

In an embodiment of the invention, w=0, or w=2 and then (W)w is selected from:

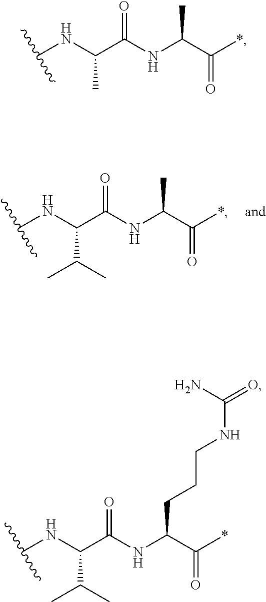

wherein
the asterisk indicates the point of attachment to (Y)_{y}; and
the wavy line indicates the point of attachment to L₂.

In an embodiment of the invention relates to an ADC wherein L is selected from:

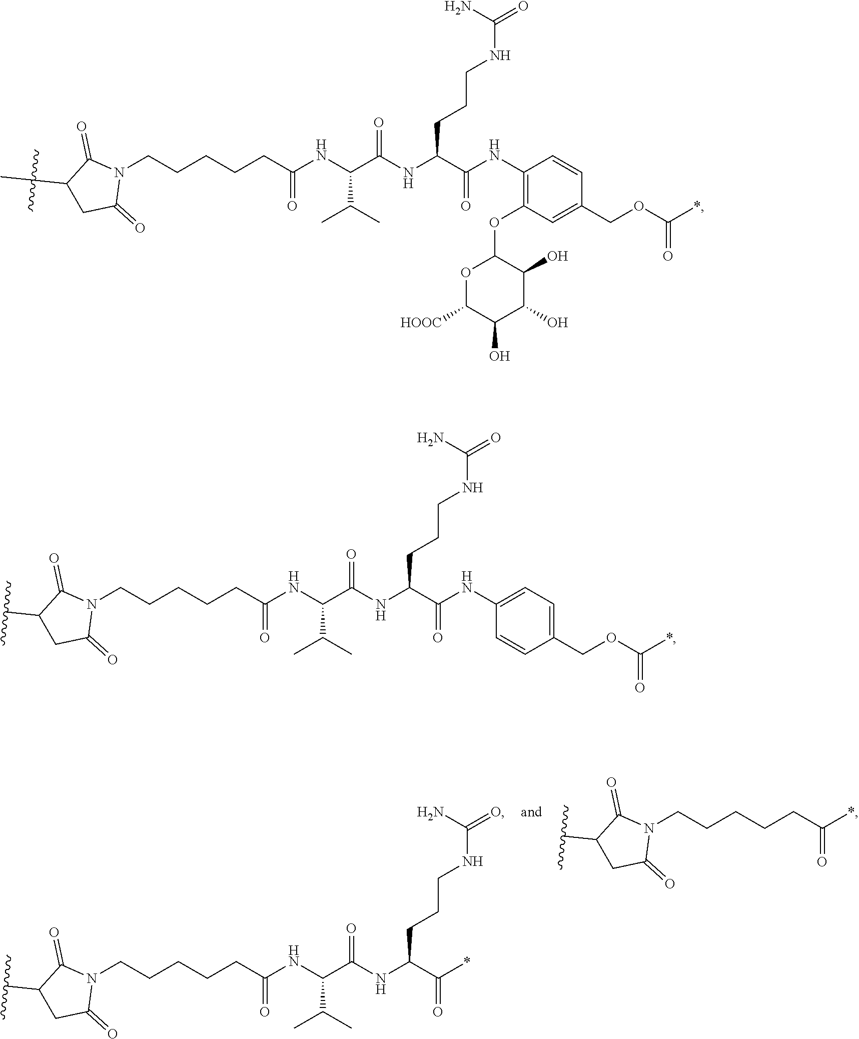

wherein the asterisk indicates the point of attachment to D. and the wavy line indicates the point of attachment to Ab.

In another embodiment the invention relates to an ADC wherein L is a linker of the following formula (III):

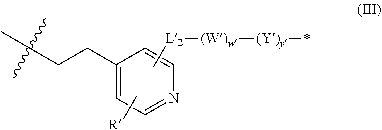

wherein
L′2 is cycloalkylene-carbonyl, (C2-C6)alkylene, or (C2-C6)alkylene-carbonyl;
W′ is an amino acid unit;
w′ is an integer of 0 to 5;
Y′ is PAB-carbonyl with PAB being

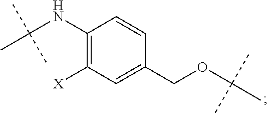

x can be H or

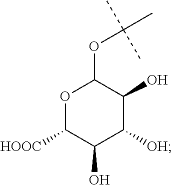

y′ is 0 or 1;
R′ is C₁-C₃ alkenyl or H.

In an embodiment the compound of formula (III) is a compound of formula (III′):

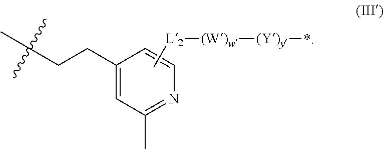

In an embodiment the compound of formula (III′) is characterised with L₂′ being C₂ alkylene carbonyl and w′ being 2.

In an embodiment the linker compound of formula (III′) is:

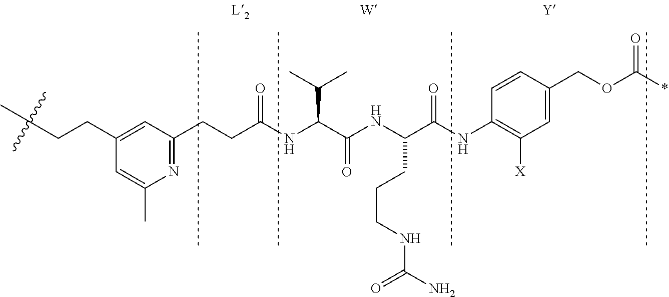

In another embodiment, the linker compound of formula (III′) is:

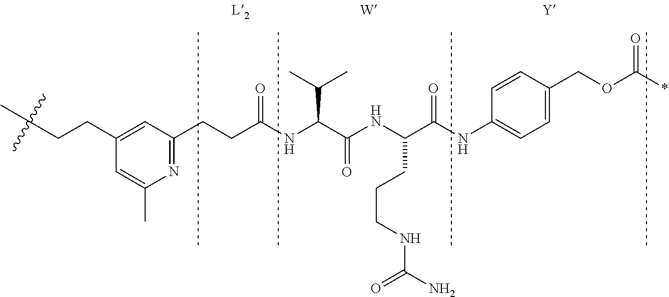

In another embodiment, the linker compound of formula (III′) is:

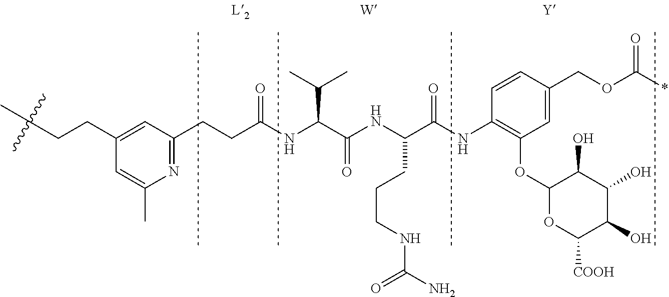

The present linkers may be synthesized using amide bond coupling. Many methods exist for amide synthesis. Some methods, but not limited to, are described in Montalbetti, Christian A. G. N (Tetrahedron 61(46), 2005, 10827-10852). Alternatively, the linkers may be synthesized using standard stepwise addition of one or more residues using, for example, a peptide or protein synthesizer. Alternatively, other methods that may be used for amide formation includes, but not limited to, Beckmann rearrangement, Schmidt reaction, Nitrile hydrolysis, Willgerodt-Kindler reaction, Passerini reaction, Ugi reaction, Bodroux reaction, Chapman rearrangement, Leuckart amide synthesis, Ritter reaction, Ester aminolysis, Schotten-Baumann reaction, ruthenium-based catalysis of alcohol and amine, or Photolytic addition of formamide to olefins.

In one embodiment, the cytotoxin is selected from the group consisting of monomethyl auristatin E (MMAE), monomethyl auristatin F (MMAF), mertansine (DM-1), saporin, gemcitabine, irinotecan, etoposide, vinblastine, pemetrexed, docetaxel, paclitaxel, platinum agents (for example, cisplatin, oxaliplatin and carboplatin), vinorelbine, capecitabine, mitoxantrone, ixabepilone, eribulin, 5-fluorouracil, trifluridine and tipiracil.

The antigen-binding molecule may also be conjugated to a detectable moiety.

Detectable moieties contemplated by the present invention include for example any species known in the art that is appropriate for diagnostic detection, including in vitro detection and in vivo imaging. The detectable moiety may be, for example, a fluorophore, a radionuclide reporter, a metal-containing nanoparticle or microparticle, an ultrasound contrast agent (e.g., a nanobubble or microbubble) or an optical imaging dye. This also includes contrast particles visible in magnetic resonance imaging (MRI) and magnetic particle imaging (MPI). Fluorophores can be detected and/or imaged, for example, by fluorescence polarization, fluorescence-activated cell sorting and fluorescence microscopy, which may or may not be in combination with electrospray ionization-mass spectrometry (ESI-MS) detection, as well as fluorescence emission computed tomography (FLECT) imaging. Radionuclide reporters can be detected and imaged by radionuclide (nuclear) detection, such as, for example, single-photon emission computed tomography (SPECT), positron emission tomography (PET) or scintigraphic imaging. Metal-containing nanoparticles or microparticles may be detected using optical imaging, including MRI, which is typically used with paramagnetic nanoparticles or microparticles, and MPI, which is generally used with superparamagnetic particles. Ultrasound contrast agents can be detected using ultrasound imaging including contrast-enhanced ultrasound (CEU).

The detectable label may also be an enzyme-substrate label. The enzyme may generally catalyse a chemical alteration of the chromogenic substrate that can be measured using various techniques. For example, the enzyme may catalyse a chemical alteration of the chromogenic substrate that can be measured using the various techniques. For example, the example may catalyse a colour change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as unease and xanthine oxidase), lactoperoxidase, microperoxidase, and the like.

Examples of enzyme-substrate combinations include, for example:
1) Horseradish peroxidase (HRP) utilizes hydrogen peroxide to oxidize a dye precursor (e.g., orthophenylene diamine (OPD) or 3,3′,5,5′-tetramethyl benzidine hydrochloride (TMB));
2) alkaline phosphatase (AP) with para-Nitrophenyl phosphate as chromogenic substrate; and
3) β-D-galactosidase (β-D-Gal) with a chromogenic substrate (e.g., p-nitrophenyl-β-D-galactosidase) or fluorogenic substrate 4-methylumbelliferyl-β-D-galactosidase.

In another embodiment of the invention, the antigen-binding molecule need not be labelled, and the presence thereof can be detected using a labeled antibody which binds to the antigen-binding molecule. The antigen-binding molecule of the present invention may be employed in any known assay method, such as competitive binding assays, direct and indirect sandwich assays, immunohistochemistry and immunoprecipitation assays.

In one embodiment, the antigen-binding molecule selectively binds to a gefitinib-resistant lung cancer cell, an osimertinib-resistant lung cancer cell, a non-small cell lung cancer cell, a breast cancer cell, a pancreatic cancer cell, a stomach (or gastric) cancer cell, a small intestine cancer cell, an oesophageal cancer cell or a colorectal cancer cell.

Disclosed herein is an isolated polynucleotide comprising a nucleic acid sequence encoding the antigen-binding molecule as defined herein.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

Also disclosed herein is a vector that comprises a nucleic acid encoding the antigen-binding molecule as described herein.

By “vector” is meant a nucleic acid molecule, preferably a DNA molecule derived, for example, from a plasmid, bacteriophage, or virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a construct comprising a polynucleotide as defined herein in operable connection with one or more control sequence.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Disclosed herein is a host cell that contains the construct as defined herein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

Disclosed herein is a pharmaceutical composition comprising an antigen-binding molecule as defined herein and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, colouring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciated that other methods known in the art to incorporate an antigen-binding molecule, as described herein, into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the antigen-binding molecule can be determined by a skilled artisan. In certain embodiments, the antigen-binding molecule is administered by injection (e.g., subcutaneously or intravenously) at a dose of about 0.01 to 50 mg/kg, e.g., 0.01 to 0.1 mg/kg, e.g., about 0.1 to 1 mg/kg, about 1 to 5 mg/kg, about 5 to 25 mg/kg, about 10 to 50 mg/kg. The dosing schedule can vary from e.g., once a week to once every 2, 3, or 4 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Disclosed herein is an antigen-binding molecule or a composition as defined herein for use as a medicament.

Disclosed herein is a method of treating or preventing cancer or inflammatory disease in a subject, the method comprising administering a therapeutically effective amount of an antigen-binding molecule or a composition as defined herein to the subject.

The terms “treating”, “treatment” and the like, are used interchangeably herein to mean relieving, reducing, alleviating, ameliorating or otherwise inhibiting the condition, including one or more symptoms of the condition. The terms “prevent”, “preventing”, “prophylaxis”, “prophylactic”, “preventative” and the like are used interchangeably herein to mean preventing or delaying the onset of the condition, or the risk of developing the condition.

The terms “treating”, “treatment” and the like also include relieving, reducing, alleviating, ameliorating or otherwise inhibiting the effects of the condition for at least a period of time. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In one embodiment, the subject is a human subject.

The terms “cancer” and “cancerous” refer to or describe the physiological condition in mammals that is typically characterized in part by unregulated cell growth. As used herein, the term “cancer” refers to non-metastatic and metastatic cancers, including early stage and late-stage cancers. By “non-metastatic” is meant a cancer that remains at the primary site and has not penetrated into the lymphatic or blood vessel system or to tissues other than the primary site. The term “metastatic cancer” refers to cancer that has spread or is capable of spreading from one part of the body to another. Generally, a non-metastatic cancer is any cancer that is a Stage 0, I, or II cancer, and occasionally a Stage III cancer. A metastatic cancer, on the other hand, is usually a Stage IV cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, oesophageal cancer, small intestine cancer, endocrine cancer, thyroid cancer, parathyroid cancer, adrenal cancer, soft tissue tumor, urethral cancer, penile cancer, prostate cancer, chronic or acute leukaemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof.

In one embodiment, the cancer cell is a solid or haematological cancer cell.

The term “haematological cancer” may refer to one or more of leukemia, lymphoma, Chronic Myeloproliferative Disorders, Langerhans Cell Histiocytosis, Multiple Myeloma/Plasma Cell Neoplasm, Myelodysplasia Syndromes, Myelodysplastic/Myeloproliferative Neoplasms or a combination thereof. In some embodiments, leukemia is any one or more of Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia (HCL) or a combination thereof. In some embodiments, lymphoma is any one or more of AIDS-Related Lymphoma, Cutaneous T-Cell Lymphoma, Hodgkin Lymphoma, Mycosis Fungoides, Non-Hodgkin Lymphoma, Primary Central Nervous System Lymphoma, Sezary Syndrome, T-Cell Lymphoma, Cutaneous, Waldenstrom Macroglobulinemia, B cell lymphoma or a combination thereof.

The term “solid cancer” may refer to one or more of breast cancer, large intestinal cancer, lung cancer, small cell lung cancer, gastric (stomach) cancer, liver cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, oesophageal cancer, small intestine cancer, endocrine cancer, thyroid cancer, parathyroid cancer, adrenal cancer, soft tissue tumor, urethral cancer, penile cancer, prostate cancer, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer, sarcomas or a combination thereof.

In one embodiment, the cancer is a metastatic cancer. The cancer may be a refractory or a relapsed cancer.

In one embodiment, the cancer is selected from the group consisting of gefitinib-resistant lung cancer, osimertinib-resistant lung cancer, non-small cell lung cancer, breast cancer, pancreatic cancer, stomach (or gastric) cancer, small intestine cancer, oesophageal cancer and colorectal cancer.

In one embodiment, the cancer is one that is associated with the overexpression of CEACAM5 and/or CEACAM6. In one embodiment, the cancer is one that is associated with the overexpression of glycosylated CEACAM5 and/or CEACAM6. In one embodiment, the cancer is one that is associated with the overexpression of glycosylated CEACAM5. In one embodiment, the cancer is one that is associated with the overexpression of glycosylated CEACAM6. In one embodiment, the cancer is one that is associated with the overexpression of glycosylated CEACAM5 and CEACAM6. The CEACAM5 and/or CEACAM6 may be glycosylated at position N256.

In one embodiment, the inflammatory disease is Crohn's disease or asthma. The asthma may be caused by neutrophilic inflammation.

The methods as disclosed herein may comprises the administration of a “therapeutically effective amount” of an agent (e.g. an antigen-binding molecule, a polynucleotide, a construct, a vector, a host cell or a pharmaceutical composition) to a subject. As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

Disclosed herein is an antigen-binding molecule or a composition as defined herein for use in treating or preventing cancer or an inflammatory disease in a subject.

Disclosed herein is the use of an antigen-binding molecule or a composition as defined herein in the manufacture of a medicament for treating or preventing cancer or an inflammatory disease in a subject.

The medicament may be administered with one or more further active pharmaceutical ingredients. In one embodiment, the medicament is to be administered with a chemotherapy.

Disclosed herein is a method for detecting a cancer or an inflammatory disease in a subject, the method comprising: contacting a sample obtained from the subject with an antigen-binding molecule as defined herein, wherein an increase in the level of binding of the antigen-binding molecule in the sample as compared to a reference is indicative of a cancer or an inflammatory disease.

Disclosed herein is a method for identifying a subject susceptible to a cancer or an inflammatory disease, the method comprising: contacting a sample obtained from the subject with an antigen-binding molecule as defined herein, wherein an increase in the level of binding in the sample as compared to a reference indicates that the subject is susceptible to cancer or an inflammatory disease.

In one embodiment, the sample is a cell, tissue or blood sample.

In one embodiment, the antigen-binding molecule comprises a detectable label. The detectable label may be selected from the group consisting of a fluorescent label, a chemiluminescent label, an enzymatic label and a radionuclide label. The detectable label may be selected from the group consisting of biotin, alkaline phosphatase, horseradish peroxidase, FITC, PE and Cy Dyes. The detectable label may be detected in an assay selected from flow cytometry, tissue section, immunofluorescence, immunocytochemistry or immunohistochemistry.

Disclosed herein is kit when used in the method as defined herein, comprising an antigen-binding molecule as defined herein, together with instructions for use.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1 Generation of Humanized Variants

GR6A04 is a previously described mouse IgG1κ monoclonal antibody developed against NSCLC, with demonstrated specificity to colorectal, gastric and breast cancer lines on flow cytometry and cancer tissue samples. It has exhibited in vitro and in vivo cytotoxic activity as an antibody drug conjugate conjugated with monomethyl auristatin E (MMAE) and as a naked antibody.

During the process of converting the murine antibody to a humanized antibody suitable for clinical therapeutic applications, it was discovered that unlike a typical antibody humanization process, many variants showed decreased specificity and/or function compared to the parental murine and chimeric forms. Several key criteria were identified to rank the humanized variants resultant from in silico humanization optimisation. Importantly, it was the aggregation and site specificity read-outs that enabled the elimination of problematic motifs, and enabled rapid short-listing of three final molecules for further pre-clinical evaluation.

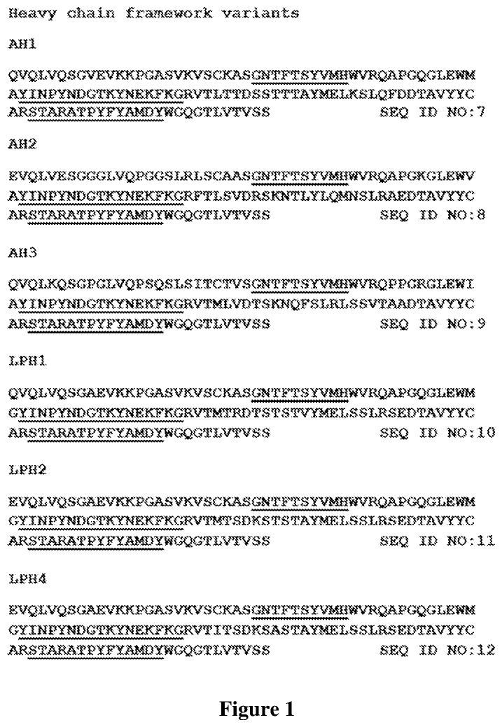

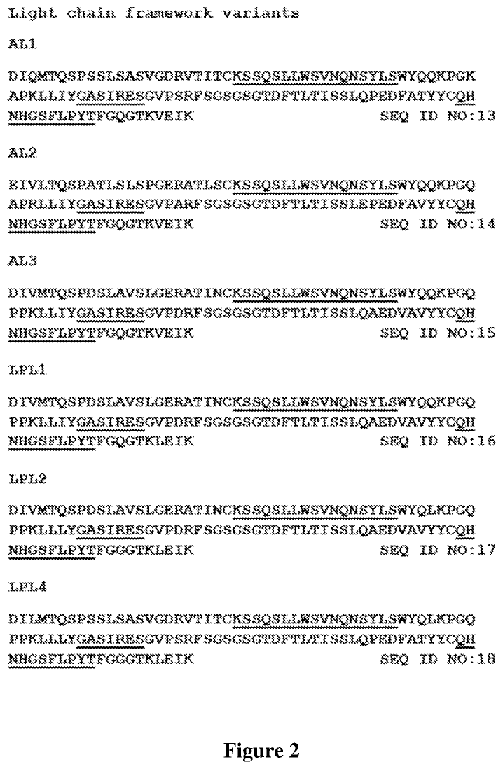

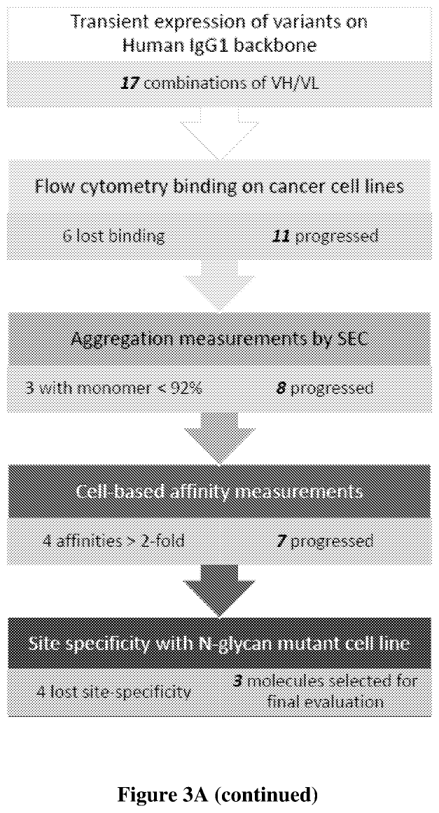

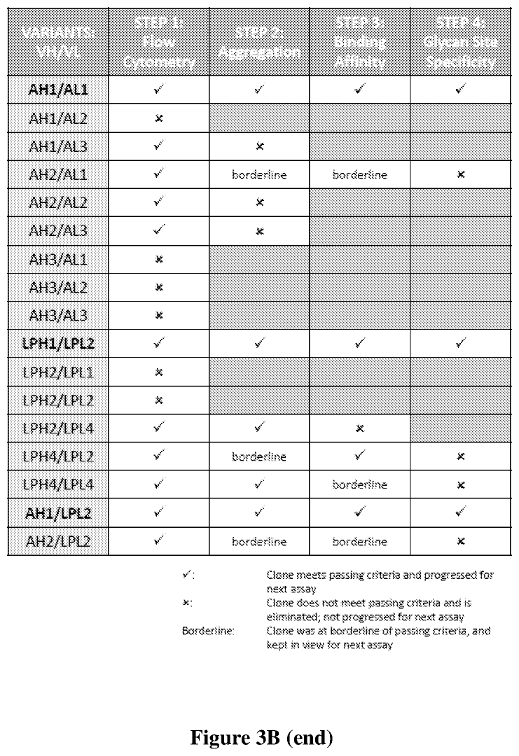

6 framework heavy chain, and 6 framework light chain sequences resulted from in silico humanization, giving rise to 17 unique heavy/light combinations (see FIG. 1-3) for the humanized variants containing the CDRs of the murine antibody, GR6A04, an anti-N-glycosylated-CEACAM5/6 therapeutic antibody, which binds, in particular, to CEACAM5/6 when glycosylated on N256.

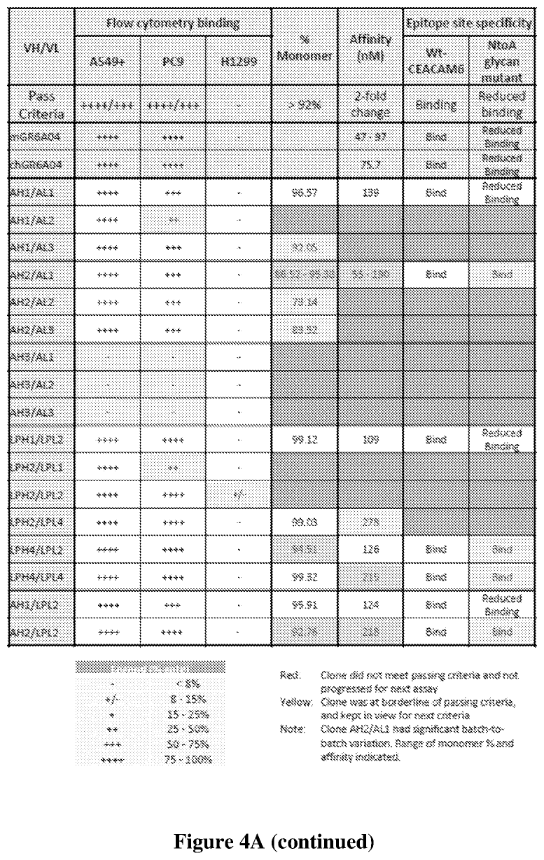

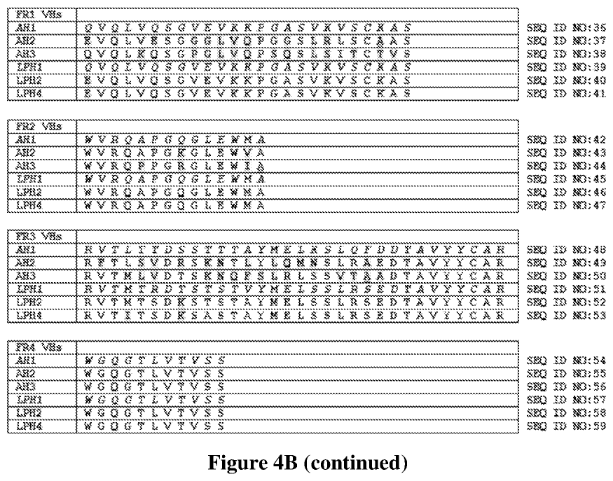

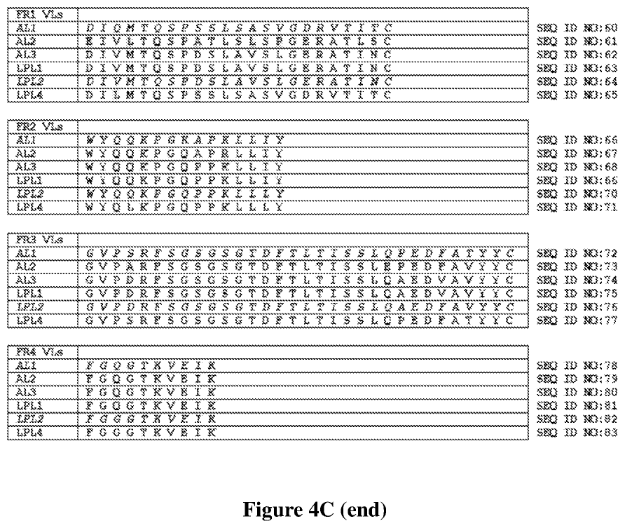

In order to screen these variants for their retention of the original functionality and downstream developability, 4 key criteria were identified, and were evaluated (see FIG. 4A) in the following order: (1) Flow cytometry binding of 3 cancer lines (2 binding and 1 non-binding); (2) Aggregation propensity by SEC; (3) Cell-based binding affinity with 1 cancer line; and (4) Epitope site specificity through the use of a CEACAM6 mutant with an N (Asparagine)-to-A (Alanine) amino acid switch at position 256 to mutate the N-glycan binding site

Importantly, unlike most standard humanization protocols whereby the grafting of the CDR to a human framework is relatively straightforward, and the screening is mainly based on binding and/or affinity, our workflow is unique in that it complements this with early developability screening with aggregation measurements by SEC, and also epitope site specificity. The epitope site specificity assay was noteworthy as it eliminated a few promising candidates that fared well on flow cytometry binding and affinity testing. It was these two criteria that give rise to critical specificity and manufacturability information that would otherwise have been overlooked in a simple binding and/or affinity screen.

Through the elimination of clones at each stage of the screening process, 3 final molecules were identified that passed all 4 key criteria, and thereby retains the most similar functionality to the original murine antibody. These 3 final molecules are AH1/AL1, LPH1/LPL2 and AH1/LPL2. Further evaluation with stably expressed humanized antibodies, and in vitro efficacy of the directly-conjugated antibody drug conjugates will allow for the shortlisting of the final lead molecule to be progressed for clinical testing.

### Example 2 Characterization of Humanized Variants

### Alignment Studies

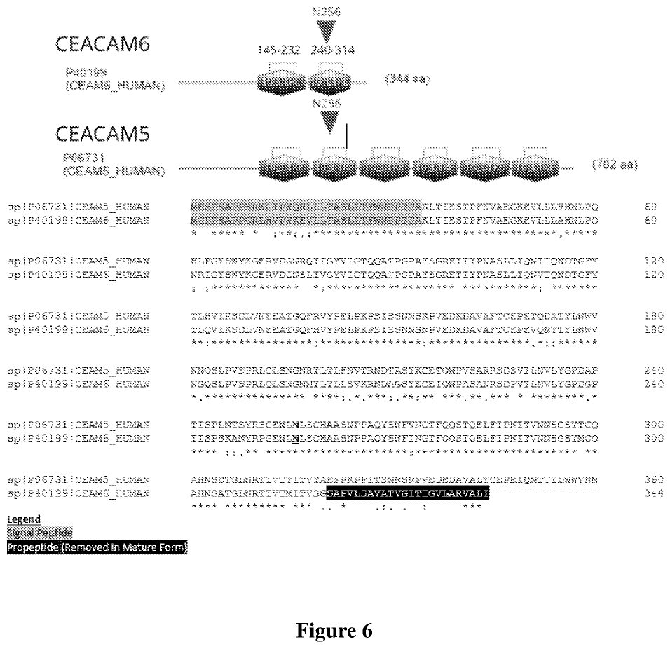

The alignment of CEACAM5 and CEACAM6 show a conserved N256 glycosylation site (FIG. 6).

### Western Blot Studies

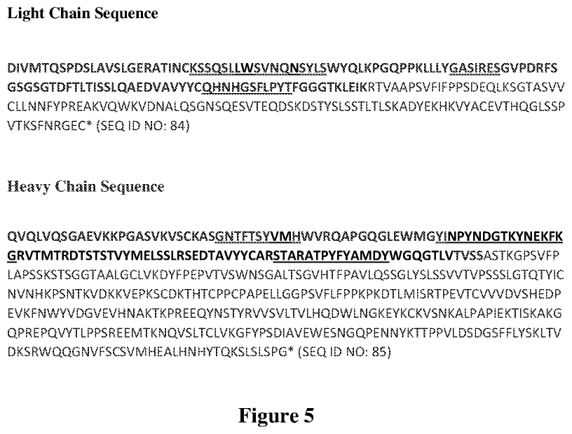

In western blot analysis using A549 lysate AB1 (with heavy and light chain sequences as shown in FIG. 5) detected 2 bands at 75 kDa and 180 kDa.

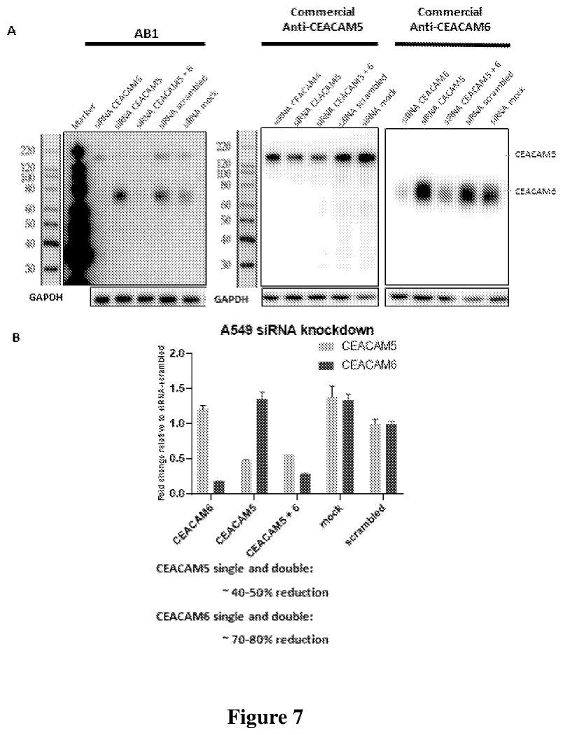

In order to determine if the 2 bands seen in Western blot are CEACAM5 and CEACAM6, A549 cells were subjected to single and double siRNA knock down of CEACAM5 and CEACAM6. Western Blot analysis of siRNA treated cell lysate show that AB1 does not bind to the 180 kDA band in CEACAM5 single or double knock down samples. Further it is shown that AB1 does not bind to the 75 kDA band in CEACAM6 single or double knock down samples (FIG. 7A). This indicates that the antigen of AB1 is CEACAM5 and CEACAM6. Efficiency of siRNA knock down was monitored by gene expression analysis (FIG. 7B). CEACAM5 was knocked-down less efficiently than CEACAM6 in single as well a double knock down samples. This explains why the commercial anti-CEACAM5 antibody still picked up CEACAM5 in the single and double knock down samples but with less intensity (FIG. 7a). Commercial anti CEACAM6 antibody detected CEACAM6 which significant less intensity in single and double knock down (FIG. 7a).

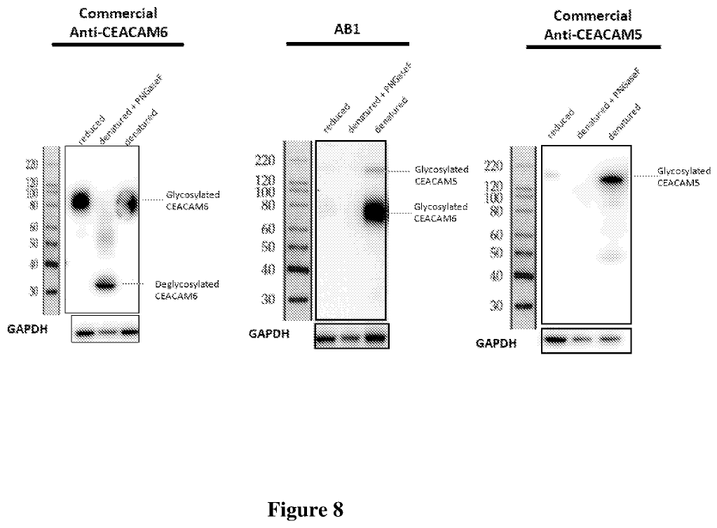

It was further shown that an N-glycosylation is important for the binding of AB1 to CEACAM5 and CEACAM6. A549 cell lysate was reduced, denatured and treated with PNGase F to remove N-linked glycans (FIG. 8). Cell lysate was probed with the AB1 antibody and commercial anti CEACAM5 and anti CEACAM6 antibodies. Western Blot analysis show that AB1 binding to the 75 kDA band (CEACAM6) and the 180 kDa band (CEACAM5) is abolished after reducing and after PNGase F treatment. Commercial anti CEACAM6 antibody detects reduced protein at 75 kDA and de-glycosylated CEACAM6 at lower size (37 kDa). The commercial anti CEACAM5 antibody does bind to reduced CEACAM5 with less intensity but seems to be also N-glycan dependent as no de-glycosylated CEACAM5 is detected. This shows that N-glycosylation is important for the binding of AB1 to CEACAM5 and CEACAM6.

### FACS Binding Analysis

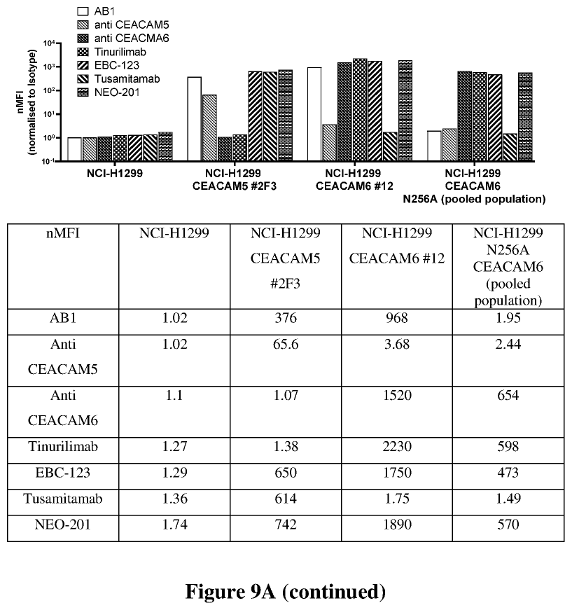

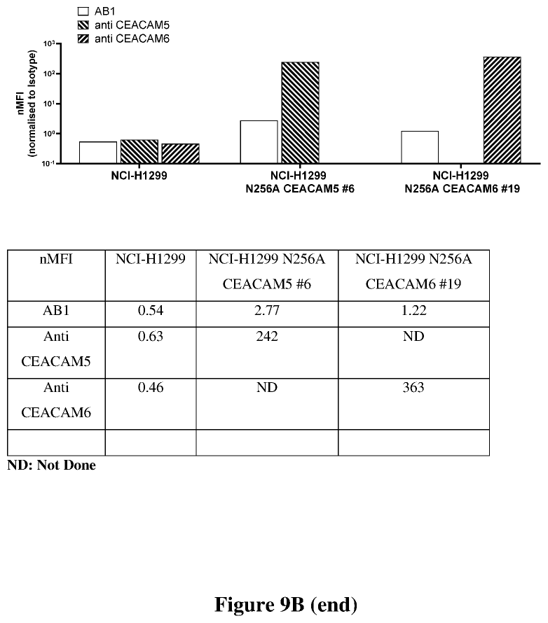

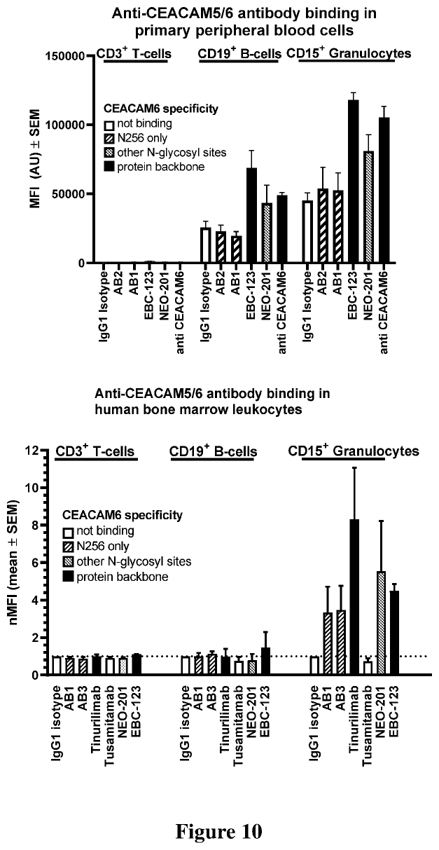

N256-glycosylation dependency of AB1 recognition was shown by Flow cytometric analysis. Flow binding analysis (FIG. 9) was performed on NCI-H1299 or NCI-H1299 overexpressing N256A mutated CEACAM5 or NCI-H1299 N256A mutated CEACAM6 or NCI-H1299 CEACAM5 or NCI-H1299 CEACAM6.

The lung cancer cell line NCI-H1299, which is not expressing CEACAM5 nor CEACAM6 was transfected with CEACAM5 or CEACAM6 sequence and a CEACAM5 or CEACAM6 mutant sequence in which the N-glycosylation position N256 was mutated (N256A, Asparagine to Alanine). This mutation abolishes N-glycosylation. Mean fluorescent intensity (MFI) was normalized (nMFI) against a human IgG1 isotype control (see Table in FIGS. 9A and B). Flow cytometry binding of AB1 was compared to commercial anti CEACAM5 and CEACAM6 antibodies as well as competitor antibodies. Competitor antibodies included biosimilars to CEACAM6 specific Tinurilimab (Bayer), CEACAM5 specific Tusamitamab (Sanofi), N-glycosylated CEACAM5 and CEACAM6 specific NEO-201 (Precision Biologics) and CEACAM5 and CEACAM6 specific EBC-123. EBC-123 is a biosimilar of L-DOS47 (Helix Biopharma Corp) whereby the original monomeric camelid single domain VHH 2A3 is engrafted on a human Fc. It was shown that AB1 does bind to NCI-H1299 cells expressing CEACAM5 or CEACAM6 (FIG. 9A), but not to cells expressing the N256A mutated CEACAM5 or N256A mutated CEACAM6 (FIG. 9B). This indicates that N256 glycosylation of CEACAM6 is important for the binding of AB1 to its antigen recognition motif. None of the CEACAM6 specific competitor antibodies (Tinurilimab, EBC-123 and NEO-201) showed this dependency on N256 glycosylation of CEACAM6 (FIG. 9A.

### Granulocyte Binding

The humanized leads are differentiated from test competitor compounds by binding less to granulocytes (CD15+ cell population) or B-cells (CD 19+ population) in the peripheral blood or bone marrow cells (FIG. 10).

Flow cytometry binding of AB1, AB2 and AB3 to different populations of primary peripheral blood cells (FIG. 10 top) or primary bone marrow leukocytes (FIG. 10 below) in comparison to competitor antibodies. Primary cells with red blood cells lysed were incubated with different primary antibodies targeting CEACAM5 and CEACAM6. Cells were labelled with fluorescent-labelled linage markers for granulocytes (CD15+), T-cells (CD3+) or B-cells (CD19+). Mean fluorescent intensity (MFI, top) or normalized MFI (nMFI) (below) is shown for 3 donor/2 repeats for primary peripheral blood cells and 1 donor/3 repeats for primary human bone marrow leukocytes. No (i.e. negligible) binding was seen to CD3⁺ T-cells by AB1, AB2 or AB3, AB1, AB2 and AB3 were found to bind less to granulocytes (CD15+ cell population) or B-cells (CD 19+ population) in the peripheral blood compared to CEACAM6 specific antibodies like Tinurilimab (Bayer), NEO-201 (Precision Biologics) and EBC-123 (Helix Biopharma Corp). The CEACAM5 specific Tusamitamab (Sanofi) shows no binding to human leukocytes.

### Affinity Studies

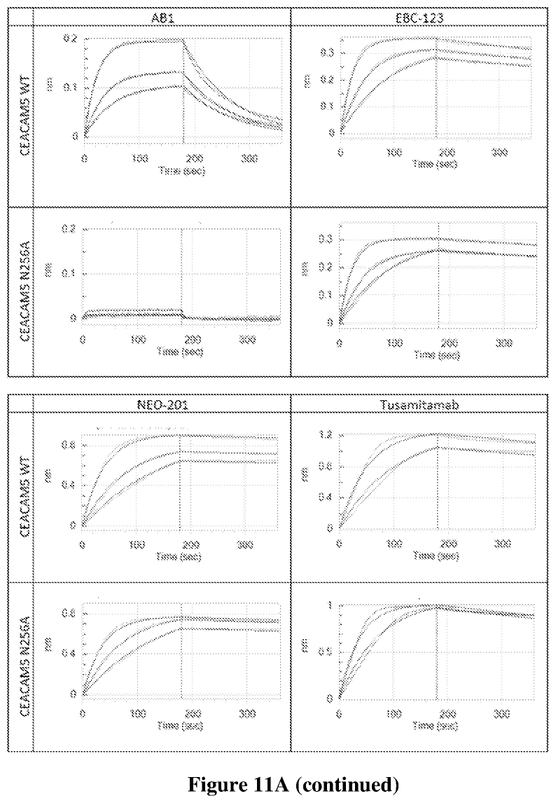

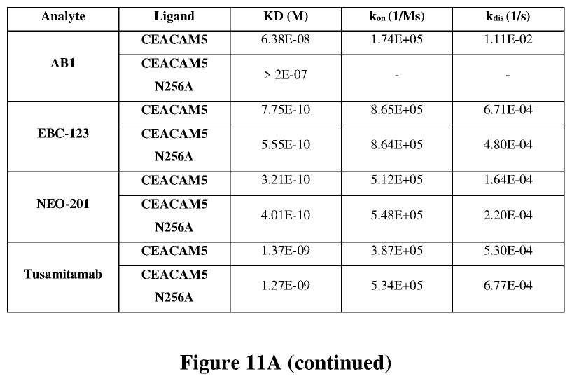

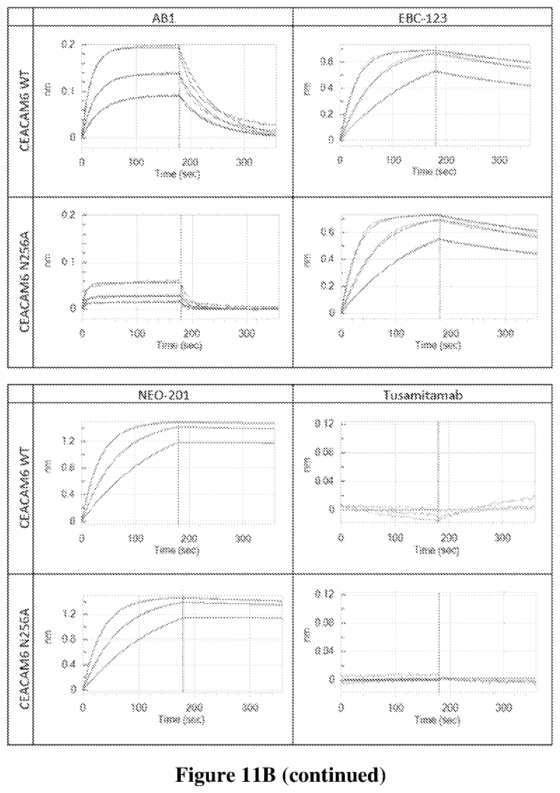

Affinity measurement for AB1 was done by Bio-layer Interferometry using an Octet 384-Red (FIG. 11). In-house produced Avi-tagged CEACAM5, CEACAM6 and N256A mutated CEACAM5 and N256A mutated CEACAM6 were immobilized to a Dip and Read SA biosensor. The antibody AB1 and competitor antibodies EBC-123 (Helix Biopharma Corp), NEO-201 (Precision Biologics) and Tusamitamab (Sanofi) were used as analyte.

Results show that AB1 binds to both CEACAM5 and CECAM6 with comparable affinity constants (KDs) in the 2-digit nanomolar range. KDs for the AB1 were 10-100-fold smaller on N256A mutated CEACAM5 and N256A mutated CEACAM6 compared to CEACAM5 and CEACAM6 protein. The competitor antibodies bound to both wildtype and mutant CEACAM5 and CEACAM6 with similar affinity.

### Internalization Studies

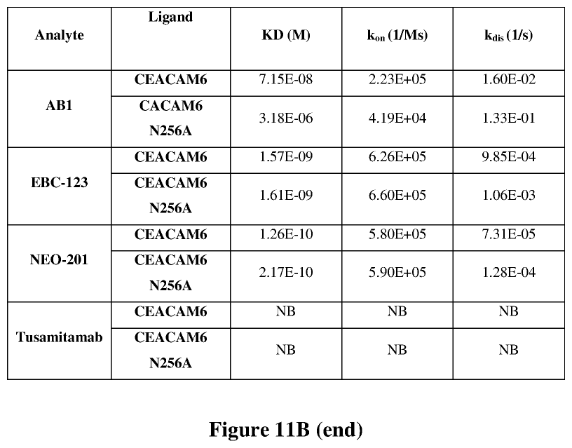

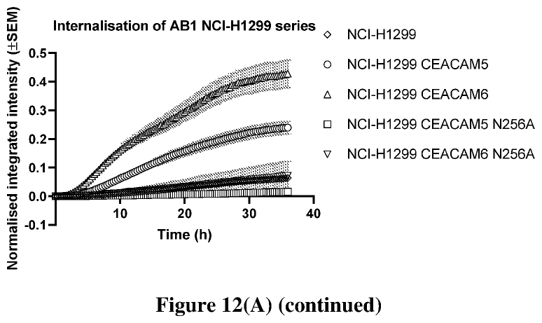

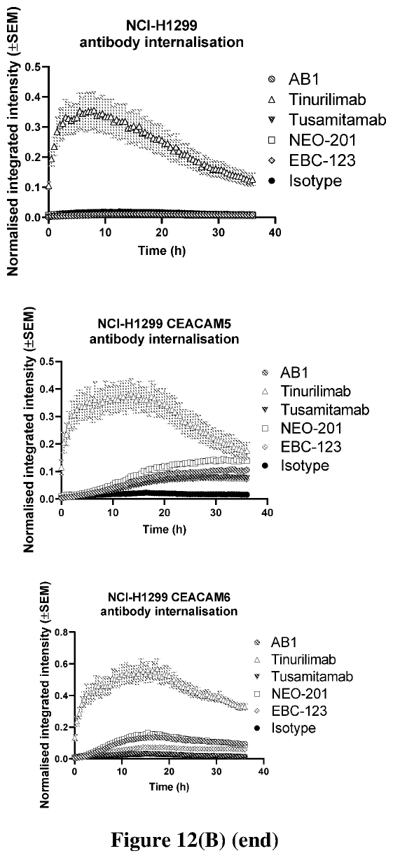

Internalization of AB1 and competitor antibodies into antigen positive and antigen negative cell lines was evaluated (FIG. 12). FIG. 12A shows internalization of AB1 in NCI-H1299 overexpressing CEACAM5 or CEACAM6 or N256A mutated CEACAM5 or N256A mutated CEACAM6. FIG. 12B) shows internalization of AB1 in comparison to the IgG1 isotype control and competitor antibodies specific to CEACAM5 and/or CEACAM6 into NCI-H1299 and NCI-H1299 overexpressing CEACAM5 or CEACAM6. Cells were incubated with antibodies and anti-human FabFluor pH Red antibody. Antibody-FabFluor pH Red complex exhibits red fluorescence at low pH. Internalization was monitored up to 24 h and integrated intensity was plotted. FIG. 12A shows that AB1 internalises in NCI-H1299 expressing CEACAM5 or CEACAM6, but not NCI-H1299 or NCI-H1299 expressing N256A mutated CEACAM5 or N256A mutated CEACAM6. FIG. 12B shows that CEACAM6 specific Tinurilimab (Bayer) internalises non-specifical into NCI-H1299 cells. CEACAM5 specific Tusamitamab (Sanofi) internalizes only into NCI-H1299 overexpressing CEACAM5 while the CEACAM5 and CEACAM6 specific NEO-201 (Precision Biologics) and EBC-123 (Helix Biopharma Corp) internalise at comparable rates as AB1 into NCI-H1299 overexpressing CEACAM5 or CEACAM6.

### In Vitro Cell Killing Data (CellTiterGlo™ Viability Assay)

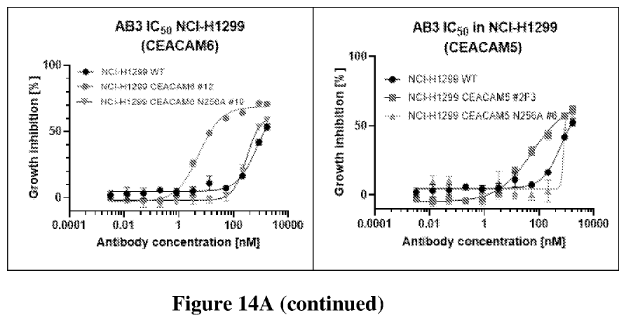

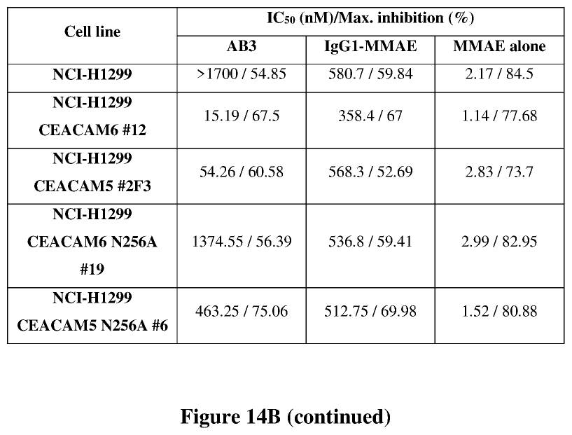

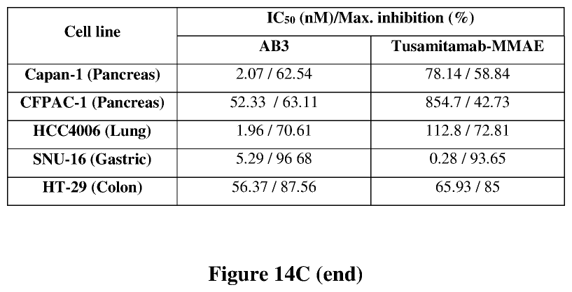

In vitro cell functionality of AB3 was demonstrated by CellTiterGlo™ viability assay and dose-response curves estimating IC₅₀ after incubation of cells for 72h. FIG. 14A left panel shows dose-response curved of AB3 with NCI-H1299 in comparison with NCI-H1299 overexpressing CEACAM6 (clonal cell line 12) or the mutated N256A CEACAM6 (clonal cell line 19). FIG. 14A) right panel shows dose-response curved of AB3 with NCI-H1299 in comparison with NCI-H1299 overexpressing CEACAM5 (clonal cell line 2F3) or the mutated N256A CEACAM5 (clonal cell line 6). FIG. 14B shows IC₅₀ obtained with AB3, IgG1 isotype control-MMAE and free MMAE with NCI-H1299 cells and NCI-H1299 cells overexpressing CEACAM5, CEACAM6, N256A mutated CEACAM5 and N256A mutated CEACAM6. 2-digit nanomolar IC₅₀ were achieved for NCI-H1299 cells that express membrane bound CEACAM5 or CEACAM6 whereas higher IC₅₀ were obtained for NCI-H1299 cells not expressing CEACAM5 nor CEACAM6 and NCI-H1299 cells expressing the N256A mutated CEACAM5 or the N256A mutated CEACAM6. Comparable high IC₅₀ were achieved with the IgG1-MMAE isotype control for all cell lines, demonstrating the specificity of AB3. Free MMAE has an IC₅₀ in the picomolar range, demonstrating the specificity of AB3. FIG. 14C shows IC₅₀ with AB3 in comparison to the CEACAM5 specific competitor antibody Tusamitamab (Sanofi) conjugated to MMAE. AB3 target a wider range of cancer indications due to its dual specificity for N256 glycosylated CEACAM5 and N256 glycosylated CEACAM6.

### In Vivo Data

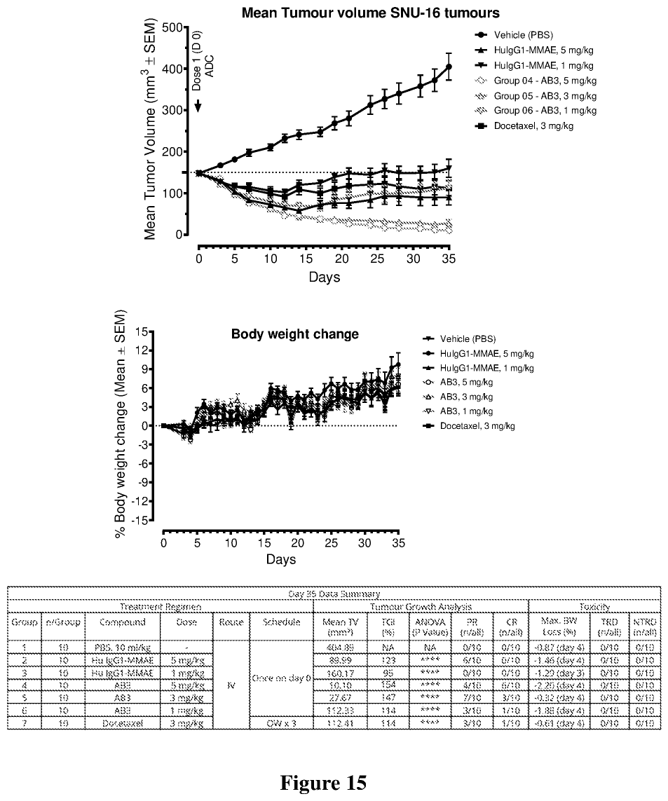

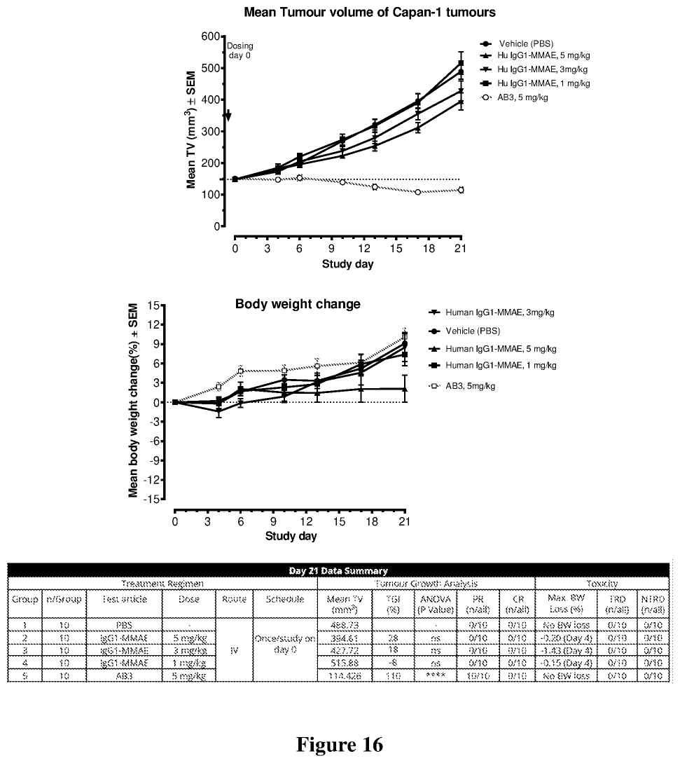

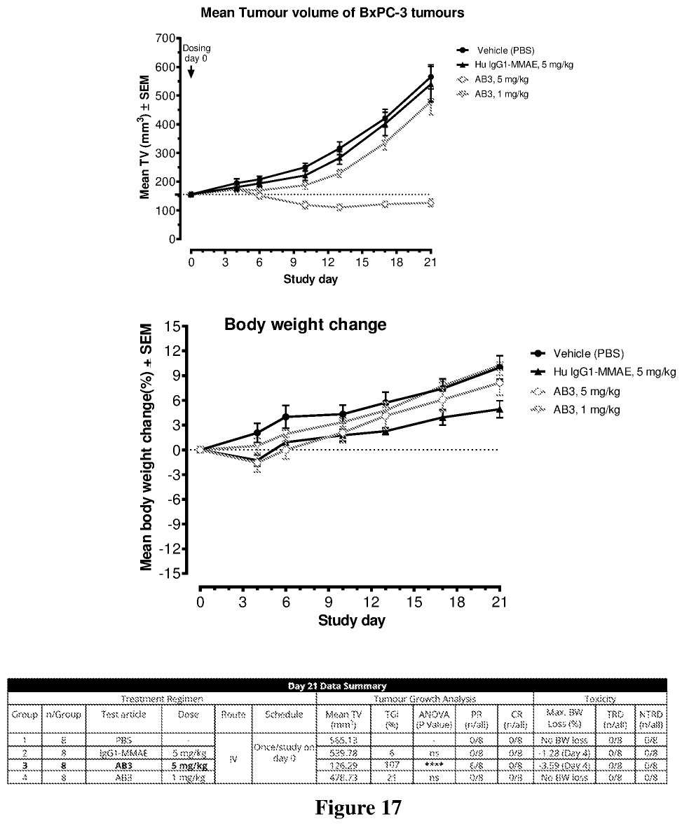

AB3 is shown in an in vivo in one gastric cancer xenograft models with SNU-16 (FIG. 15) and 2 pancreatic models (Capan-1, FIG. 16 and BxPC-3, FIG. 17). Endpoints of the efficacy study were tumour growth inhibition (TGI) observed in treatment groups, tolerability (as monitored by body weight measurements and monitoring for clinical signs) and, in some of the studies, the AB1 antigen levels on the tumour cells were also monitored throughout the study (data not shown).

SNU-16, Capan-1 and BxPC-3 cells expresses both AB1 antigens CEACAM5 and CEACAM6. Female Balb/c nude mice (n=10/group) were implanted with SNU-16 cells, Female NSG-mice (n=10/group) were implanted with Capan-1 cells and female NOD-SCID mice (n=8/group) were implanted with BxPC-3 cells. Mice were treated once IV (or once weekly x3 for docetaxel). AB3 was given at 1, 3 or 5 mg/kg for SNU-16 and Capan-1 with only a single administration on Day 0 For the BxPC-3 model the 3 mg/kg dose was omitted. AB3 achieved in SNU-16 model TGIs of 154%, 147% and 114% respectively on Day 35. Treatment with HuIgG1-MMAE (isotype control) at 5 or 1 mg/kg had statistical significantly (p=0.05) lower TGI (123% and 95% respectively). Furthermore, only AB3 treated mice showed complete responses in a dose dependent manner, with 6/10, 3/10 and 1/10 mice being tumour free after a single dose of 5, 3 and 1 mg/kg AB3 respectively, but no tumour free animals observed after treatment with HuIgG1-MMAE only (see FIG. 15).

In Capan-1 model AB3 achieved TGIs of 110% on Day 21 Treatment with HuIgG1-MMAE at 5, 3 or 1 mg/kg had statistical significantly (p=0.05) lower TGI (28%, 185 and −8% respectively) (see FIG. 16).

In BxPC-3 model AB3 achieved TGIs of 107% and 21% respectively on Day 21. Treatment with HuIgG1-MMAE at 5 mg/kg had statistical significantly (p=0.05) lower TGI of 6%. Treatment was very well tolerated with no weight loss observed (see FIG. 17).

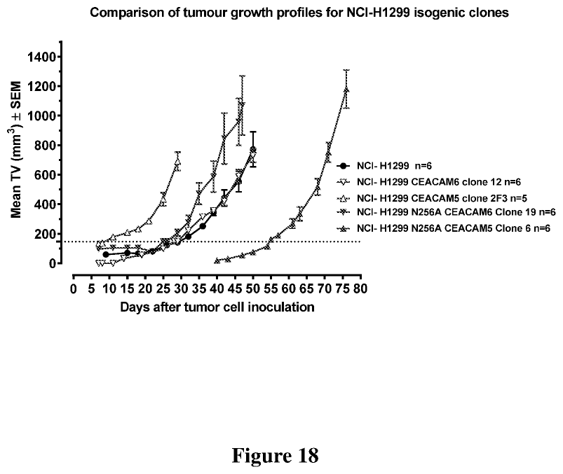

The expression of CEACAM5, CEACAM6, N256A mutated CEACAM5 and N256A mutated CEACAM6 on tumour growth was demonstrated (see FIG. 18). Cells were implanted into female NOD-SCID mice and tumour volumes were measured 3 times per week post randomization in two dimensions using a caliper. Volume was expressed in mm³ using the formula: V=(L×W×W)/2, where V is tumour volume, L is tumour length (the longest tumour dimension) and W is tumour width (the longest tumour dimension perpendicular to L). Tumour volume was measured up to 75 days and presented in FIG. 18. No difference in tumour onset or doubling time was observed for NCI-H299, NCI-H1299 overexpressing CEACAM6 or NCI-H1299 overexpressing N256A mutated CEACAM6. Early tumour onset was observed for NCI-H1299 overexpressing CEACAM5, while tumour onset for NCI-H1299 overexpressing N256A mutated CEACAM5 was delayed by 35 days. After tumour onset the tumour doubling time was comparable for all cell lines.

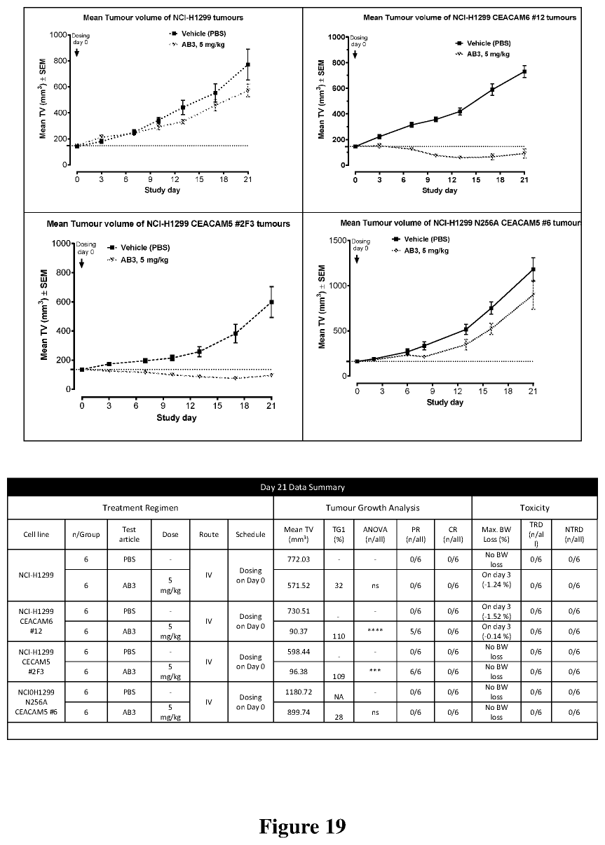

In vivo efficacy was shown in a lung cancer xenograft model using NCI-H1299 cell line and NCI-H1299 overexpressing CEACAM5 or CEACAM6 or N256A mutated CEACAM5 (see FIG. 19). Female NOD-SCID mice (n=6/group) were implanted with NCI-H1299 or NCI-H1299 overexpressing N256A mutated CEACAM5 or CEACAM5 or CEACAM6 cells and treated once IV. AB3 was given at 5 mg/kg with only a single administration on Day 0. AB3 achieved TGIs of 32% and 28% respectively for antigen negative cells NCI-H1299 and NCI-H1299 overexpressing N256A mutated CEACAM5 on Day 21. AB3 achieved TGIs of 109% and 110% respectively for antigen positive cells NCI-H1299 overexpressing CEACAM5 or CEACAM6 (see FIG. 19). Treatment was very well tolerated with no weight loss observed.

1. An antigen-binding molecule comprising:
(1) a heavy chain variable region (VH) comprising the VHCDR1 amino acid sequence of SEQ ID NO: 1, the VHCDR2 amino acid sequence of SEQ ID NO: 2, and the VHCDR3 amino acid sequence of SEQ ID NO: 3, and a light chain variable region (VL) comprising the VLCDR1 amino acid sequence of SEQ ID NO: 4, the VLCDR2 amino acid sequence of SEQ ID NO: 5, and the VLCDR3 amino acid sequence of SEQ ID NO: 6;
wherein the VH as defined in (1) comprises at least 90% sequence identity to at least one region other than a CDR of the VH amino acid sequence set forth in SEQ ID NO: 10 or 7, and the VL as defined in (1) comprises at least 90% sequence identity to at least one region other than a CDR of the VL amino acid sequence set forth in SEQ ID NO: 17 or 13.

2. The antigen-binding molecule of claim 1, wherein:
a) the VH as defined in (1) comprises at least 90% sequence identity to at least one region other than a CDR of the VH amino acid sequence set forth in SEQ ID NO: 10, and the VL as defined in (1) comprises at least 90% sequence identity to at least one region other than a CDR of the VL amino acid sequence set forth in SEQ ID NO: 17;
b) the VH as defined in (1) comprises at least 90% sequence identity to at least one region other than a CDR of the VH amino acid sequence set forth in SEQ ID NO: 10, and the VL as defined in (1) comprises at least 90% sequence identity to at least one region other than a CDR of the VL amino acid sequence set forth in SEQ ID NO: 13;
c) the VH as defined in (1) comprises at least 90% sequence identity to at least one region other than a CDR of the VH amino acid sequence set forth in SEQ ID NO: 7, and the VL as defined in (1) comprises at least 90% sequence identity to at least one region other than a CDR of the VL amino acid sequence set forth in SEQ ID NO: 17; or
d) the VH as defined in (1) comprises at least 90% sequence identity to at least one region other than a CDR of the VH amino acid sequence set forth in SEQ ID NO: 7, and the VL as defined in (1) comprises at least 90% sequence identity to at least one region other than a CDR of the VL amino acid sequence set forth in SEQ ID NO: 13.

3. The antigen-binding molecule of any one of claim 1 or 2, wherein:
a) the VH is distinguished from the VH amino acid sequence set forth in SEQ ID NO: 10 by a deletion, substitution or addition of one or more amino acids in at least one region other than a CDR of the VH amino acid sequence set forth in SEQ ID NO: 10, and the VL is distinguished from the VL amino acid sequence set forth in SEQ ID NO: 17 by a deletion, substitution or addition of one or more amino acids in at least one region other than a CDR of the VL amino acid sequence set forth in SEQ ID NO:17;
b) the VH is distinguished from the VH amino acid sequence set forth in SEQ ID NO: 10 by a deletion, substitution or addition of one or more amino acids in at least one region other than a CDR of the VH amino acid sequence set forth in SEQ ID NO: 10, and the VL is distinguished from the VL amino acid sequence set forth in SEQ ID NO: 13 by a deletion, substitution or addition of one or more amino acids in at least one region other than a CDR of the VL amino acid sequence set forth in SEQ ID NO:13;
c) the VH is distinguished from the VH amino acid sequence set forth in SEQ ID NO: 7 by a deletion, substitution or addition of one or more amino acids in at least one region other than a CDR of the VH amino acid sequence set forth in SEQ ID NO: 7, and the VL is distinguished from the VL amino acid sequence set forth in SEQ ID NO: 17 by a deletion, substitution or addition of one or more amino acids in at least one region other than a CDR of the VL amino acid sequence set forth in SEQ ID NO:17; or
d) the VH is distinguished from the VH amino acid sequence set forth in SEQ ID NO: 7 by a deletion, substitution or addition of one or more amino acids in at least one region other than a CDR of the VH amino acid sequence set forth in SEQ ID NO: 7, and the VL is distinguished from the VL amino acid sequence set forth in SEQ ID NO: 13 by a deletion, substitution or addition of one or more amino acids in at least one region other than a CDR of the VL amino acid sequence set forth in SEQ ID NO: 13.

4. The antigen-binding molecule of any one of claims 1-3, wherein the antigen-binding molecule comprises:
a) a VHFR1 that is distinguished from a VHFR1 amino acid sequence set forth in QVQLVQSGVEVKKPGASVKVSCKAS (SEQ ID NO: 19) or QVQLVQSGAEVKKPGASVKVSCKAS (SEQ ID NO: 20) by a deletion, substitution or addition of one or more amino acids;
b) a VHFR2 that is distinguished from a VHFR2 amino acid sequence set forth in WVRQAPGQGLEWMA (SEQ ID NO: 21) or WVRQAPGQGLEWMG (SEQ ID NO: 22) by a deletion, substitution or addition of one or more amino acids;
c) a VHFR3 that is distinguished from a VHFR3 amino acid sequence set forth in RVTLTTDSSTTTAYMELKSLQFDDTAVYYCAR (SEQ ID NO: 23) or RVTMTRDTSTSTVYMELSSLRSEDTAVYYCAR (SEQ ID NO: 24) by a deletion, substitution or addition of one or more amino acids;
d) a VHFR4 that is distinguished from a VHFR4 amino acid sequence set forth in YWGQGTLVTVSS (SEQ ID NO: 25) by a deletion, substitution or addition of one or more amino acids;
e) a VLFR1 that is distinguished from a VLFR1 amino acid sequence set forth in DIQMTQSPSSLSASVGDRVTITC (SEQ ID NO: 26) or DIVMTQSPDSLAVSLGERATINC (SEQ ID NO: 27) by a deletion, substitution or addition of one or more amino acids;
f) a VLFR2 that is distinguished from a VLFR2 amino acid sequence set forth in WYQQKPGKAPKLLIY (SEQ ID NO: 28) or WYQLKPGQPPKLLLY (SEQ ID NO: 29) by a deletion, substitution or addition of one or more amino acids;
g) a VLFR3 that is distinguished from a VLFR3 amino acid sequence set forth in GVPSRFSGSGSGTDFTLTISSLQPEDFATYYC (SEQ ID NO: 30) or GVPDRFSGSGSGTDFTLTISSLQAEDVAVYYC (SEQ ID NO: 31) by a deletion, substitution or addition of one or more amino acids; and/or
h) a VLFR4 that is distinguished from a VLFR4 amino acid sequence set forth in FGQGTKVEIK (SEQ ID NO: 32) or FGGGTKLEIK (SEQ ID NO: 33) by a deletion, substitution or addition of one or more amino acids.

5. The antigen-binding molecule of any one of claims 1-4, wherein the antigen-binding molecule comprises:
a) a VHFR1 amino acid sequence of QVQLVQSGX₁EVKKPGASVKVSCKAS, wherein X₁ is V or A (SEQ ID NO: 34);
b) a VHFR2 amino acid sequence of WVRQAPGQGLEWMX₂, wherein X₂ is A or G (SEQ ID NO: 35);
c) a VHFR3 amino acid sequence of RVTLTTDSSTTTAYMELKSLQFDDTAVYYCAR (SEQ ID NO: 23) or RVTMTRDTSTSTVYMELSSLRSEDTAVYYCAR (SEQ ID NO: 24);
d) a VHFR4 amino acid sequence of YWGQGTLVTVSS (SEQ ID NO: 25);
e) a VLFR1 amino acid sequence of DIQMTQSPSSLSASVGDRVTITC (SEQ ID NO: 26) or DIVMTQSPDSLAVSLGERATINC (SEQ ID NO: 27);
f) a VLFR2 amino acid sequence of WYQQKPGKAPKLLIY (SEQ ID NO: 28) or WYQLKPGQPPKLLLY (SEQ ID NO: 29);
g) a VLFR3 amino acid sequence of GVPSRFSGSGSGTDFTLTISSLQPEDFATYYC (SEQ ID NO: 30) or GVPDRFSGSGSGTDFTLTISSLQAEDVAVYYC (SEQ ID NO: 31); and/or
h) a VLFR4 amino acid sequence of FGQGTKVEIK (SEQ ID NO: 32) or FGGGTKLEIK (SEQ ID NO: 33).

6. The antigen-binding molecule of any one of claims 1-5, wherein the antigen-binding molecule comprises a VH amino acid sequence of SEQ ID NO: 10 or 7 and a VL amino acid sequence of SEQ ID NO: 17 or 13.

7. The antigen-binding molecule of any one of claims 1-6, wherein
a) the antigen-binding molecule comprises a VH amino acid sequence of SEQ ID NO: 10 and a VL amino acid sequence of SEQ ID NO: 17;
b) the antigen-binding molecule comprises a VH amino acid sequence of SEQ ID NO: 10 and a VL amino acid sequence of SEQ ID NO: 13;
c) the antigen-binding molecule comprises a VH amino acid sequence of SEQ ID NO: 7 and a VL amino acid sequence of SEQ ID NO: 17; or
d) the antigen-binding molecule comprises a VH amino acid sequence of SEQ ID NO: 7 and a VL amino acid sequence of SEQ ID NO: 13.

8. The antigen-binding molecule of any one of claims 1-7, wherein the antigen-binding molecule is an antibody or antigen-binding fragment thereof.

9. The antigen-binding molecule of claim 8, wherein the antibody or antigen binding fragment thereof is a full-length antibody, a substantially intact antibody, a Fab fragment, a scFab, a Fab′, a single chain variable fragment (scFv) or a one-armed antibody.

10. The antigen-binding molecule of any one of claims 1-9, wherein the antigen-binding molecule comprises a light chain sequence having at least 70% sequence identity to SEQ ID NO: 84, and a heavy chain sequence having at least 70% sequence identity to SEQ ID NO: 85.

11. The antigen-binding molecule of any one of claims 1-10, wherein the antigen-binding molecule binds to CEACAM5 and/or CEACAM6.

12. The antigen-binding molecule of any one of claims 1-11, wherein the antigen-binding molecule comprises is conjugated to a radioisotope or cytotoxin.

13. The antigen-binding molecule of claim 12, wherein the cyctotoxin is selected from the group consisting of monomethyl auristatin E (MMAE), monomethyl auristatin F (MMAF), mertansine (DM-1), saporin, gemcitabine, irinotecan, etoposide, vinblastine, pemetrexed, docetaxel, paclitaxel, platinum agents (for example, cisplatin, oxaliplatin and carboplatin), vinorelbine, capecitabine, mitoxantrone, ixabepilone, eribulin, 5-fluorouracil, trifluridine and tipiracil.

14. The antigen-binding molecule of any one of claims 1-13, wherein the antigen-binding molecule selectively binds to a gefitinib-resistant lung cancer cell, an osimertinib-resistant lung cancer cell, a non-small cell lung cancer cell, a breast cancer cell, pancreatic cancer cell, stomach (or gastric) cancer cell, small intestine cancer cell, esophageal cancer cell or a colorectal cancer cell.

15. An isolated polynucleotide comprising a nucleic acid sequence encoding the antigen-binding molecule according to any one of claims 1-14.

16. A construct comprising a polynucleotide of claim 15 in operable connection with one or more control sequence.

17. A host cell that contains the construct of claim 16.

18. A composition comprising an antigen-binding molecule of any one of claims 1-14 and a pharmaceutically acceptable carrier.

19. An antigen-binding molecule of any one of claims 1-14 or a composition of 18 for use as a medicament.

20. A method of treating or preventing a cancer or an inflammatory disease in a subject, the method comprising administering a therapeutically effective amount of an antigen-binding molecule of any one of claims 1-14 or a composition of 18 to the subject.

21. The method of claim 20, wherein the cancer is selected from the group consisting of gefitinib-resistant lung cancer, osimertinib-resistant lung cancer, non-small cell lung cancer, breast cancer, pancreatic cancer, stomach (or gastric) cancer, small intestine cancer, oesophageal cancer and colorectal cancer.

22. The method of claim 20, wherein the inflammatory disease is Crohn's disease or asthma.

23. An antigen-binding molecule of any one of claims 1-14 or a composition of claim 18 for use in treating or preventing cancer in a subject.

24. Use of an antigen-binding molecule of any one of claims 1-14 or a composition of claim 18, in the manufacture of a medicament for treating or preventing cancer.

25. A method for detecting cancer in a subject, the method comprising: contacting a sample obtained from the subject with an antigen-binding molecule of any one of claims 1-14, wherein an increase in the level of binding of the antigen-binding molecule in the sample as compared to a reference is indicative of cancer.

26. A method for identifying a subject susceptible to cancer the method comprising: contacting a sample obtained from the subject with an antigen-binding molecule of any one of claims 1-14, wherein an increase in the level of binding in the sample as compared to a reference indicates that the subject is susceptible to cancer.

27. The method according to claim 25 or 26, wherein the antigen-binding molecule comprises a detectable label.

28. A kit when used in the method of any one of claims 25-27, comprising an antigen-binding molecule of any one of claims 1-14, together with instructions for use.
